(19) United States
(12) Patent Application Publication
(10) Pub. No.: US 2022/0195406 A1
(43) Pub. Date: Jun. 23, 2022

(54) CRISPR/CAS-BASED GENOME EDITING COMPOSITION FOR RESTORING DYSTROPHIN FUNCTION

(71) Applicant: Duke University, Durnam, NC (US)

(72) Inventors: Charles A. Gersbach, Chapel Hill, NC (US); Adrian Pickar Oliver, Rougemont, NC (US)

(21) Appl. No.: 17/603,329

(22) PCT Filed: Apr. 14, 2020
(86) PCT No.: PCT/US2020/028154

§ 371 Date: Oct. 12, 2021

Related U.S. Application Data
(60) Provisional application No. 62/833,759, filed on Apr. 14, 2019.

(51) Int. Cl. C12N 9/22; C12N 15/11; C07K 14/47; C12N 15/90; A61K 38/46; A61K 31/7088; C12N 15/86

(52) U.S. Cl. CPC C12N 9/22; C12N 15/11; C07K 14/4708; C12N 15/907; A61K 38/465; C12N 2750/14143; C12N 15/86; C12N 2800/80; C12N 2310/20; C07K 2319/00; A61K 31/7088

(57) ABSTRACT

Disclosed herein are CRISPR/Cas-based genome editing compositions and methods for treating Duchenne Muscular Dystrophy by restoring dystrophin function.

### CROSS-REFERENCE TO RELATED APPLICATIONS

This application claims priority to U.S. Provisional Patent Application No. 62/833,759, filed Apr. 14, 2019, which is incorporated herein by reference in its entirety.

### STATEMENT REGARDING FEDERALLY SPONSORED RESEARCH

This invention was made with government support under grant R01AR069085 awarded by the National Institutes of Health. The government has certain rights in the invention.

### TECHNICAL FIELD

The present disclosure is directed to CRISPR/Cas-based genome editing compositions and methods for treating Duchenne Muscular Dystrophy by restoring dystrophin function.

### INTRODUCTION

Duchenne muscular dystrophy (DMD) is the most prevalent lethal heritable childhood disease occurring in ˜1:5000 newborn males. Progressive muscle weakness leading to mortality in patients' mid-20s is a result of mutations in the dystrophin gene. In most cases (˜60%), the mutations consist of deletions in one or more of the 79 exons from the dystrophin gene, leading to disruption of the reading frame. Previous therapeutic strategies typically aim to generate expression of a truncated but partially functional dystrophin protein that recapitulates a genotype corresponding to Becker muscular dystrophy, which is associated with milder symptoms relative to DMD. For example, several groups have adapted the CRISPR/Cas9 technology for gene editing in cultured human DMD cells and the mdx mouse model of DMD to restore the dystrophin reading frame by deleting specific exons. However, there remains a need to develop gene editing strategies to restore the complete, fully functional dystrophin protein.

### SUMMARY

In an aspect, the disclosure relates to a CRISPR/Cas-based genome editing system. The system may include one or more vectors encoding a composition, the composition comprising: (a) a guide RNA (gRNA) targeting a fragment of a mutant dystrophin gene; (b) a Cas protein or a fusion protein comprises the Cas protein; and (c) a donor sequence comprising a fragment of a wild-type dystrophin gene. In a further aspect, the system may include (a) a guide RNA (gRNA) targeting a fragment of a mutant dystrophin gene; (b) a Cas protein or a fusion protein comprises the Cas protein; and (c) a donor sequence comprising a fragment of a wild-type dystrophin gene. In some embodiments, the fragment of the wild-type dystrophin gene is flanked by two gRNA spacers and/or PAM sequences. In some embodiments, the gRNA targets an intron that is juxtaposed with an exon of the mutant dystrophin gene, and wherein the exon is selected from exons 1-8, 10, 11, 12, 14, 16-22, 43-59, and 61-66 of the mutant dystrophin gene. In some embodiments, the donor sequence comprises an exon of the wild-type dystrophin gene or a functional equivalent thereof, and wherein the exon is selected from exons 1-8, 10, 11, 12, 14, 16-22, 43-59, and 61-66 of the wild-type dystrophin gene. In some embodiments, the exon of the mutant dystrophin gene is mutated or at least partially deleted from the dystrophin gene or genome, or wherein the exon of the mutant dystrophin gene is deleted and the intron is juxtaposed to where the deleted exon would be in a corresponding wild-type dystrophin gene. In some embodiments, the exon is exon 52. In some embodiments, the gRNA binds and targets a polynucleotide sequence comprising: a) SEQ ID NO: 17 or SEQ ID NO: 18; b) a fragment of SEQ ID NO: 17 or SEQ ID NO: 18; c) a complement of SEQ ID NO: 17 or SEQ ID NO: 18, or fragment thereof; d) a nucleic acid that is substantially identical to SEQ ID NO: 17 or SEQ ID NO: 18, or complement thereof; or e) a nucleic acid that hybridizes under stringent conditions to SEQ ID NO: 17 or SEQ ID NO: 18, complement thereof, or a sequence substantially identical thereto. In some embodiments, the gRNA comprises or is encoded by a polynucleotide sequence of SEQ ID NO: 19 or SEQ ID NO: 20, or variant thereof. In some embodiments, the Cas protein is a Streptococcus pyogenes Cas9 protein or a Staphylococcus aureus Cas9 protein. In some embodiments, the Cas protein comprises an amino acid sequence of SEQ ID NO: 1, 2, 3, or 4. In some embodiments, the two gRNA spacers independently comprise a sequence selected from SEQ ID NO: 5-8 and 25-45. In some embodiments, the two gRNA spacers are identical. In some embodiments, the two gRNA spacers are different. In some embodiments, at least one of the two gRNA spacers comprises a sequence of SEQ ID NO: 25 or SEQ ID NO: 26. In some embodiments, the donor sequence comprises the polynucleotide of SEQ ID NO: 21 or SEQ ID NO: 22. In some embodiments, the vector is a viral vector. In some embodiments, the vector is an Adeno-associated virus (AAV) vector. In some embodiments, the AAV vector is AAV1, AAV2, AAV3, AAV4, AAV5, AAV6, AAV7, AAV8, AAV9, AAV-10, AAV-11, AAV-12, AAV-13, or AAVrh.74 vector. In some embodiments, one of the one or more vectors comprises the polynucleotide sequence of SEQ ID NO: 23 or 24. In some embodiments, the molar ratio between gRNA and donor sequence is 1:1, or 1:15, or from 5:1 to 1:10, or from 1:1 to 1:5.

In a further aspect, the disclosure relates to recombinant polynucleotide encoding a donor sequence comprising a fragment of a wild-type dystrophin gene or a functional equivalent thereof, and wherein the fragment or functional equivalent thereof is flanked by two gRNA spacers. In some embodiments, the donor sequence comprises an exon of the dystrophin gene, and wherein the exon is selected from exons 1-8, 10, 11, 12, 14, 16-22, 43-59, and 61-66. In some embodiments, the recombinant polynucleotide comprises a sequence of SEQ ID NO: 23 or 24.

Another aspect of the disclosure provides a vector comprising the recombinant polynucleotide as detailed herein. In some embodiments, the vector comprises a heterologous promoter driving expression of the recombinant polynucleotide.

Another aspect of the disclosure provides a cell comprising the recombinant polynucleotide as detailed herein or the vector as detailed herein.

Another aspect of the disclosure provides a composition for restoring dystrophin function in a cell having a mutant dystrophin gene, the composition comprising the system as detailed herein, the recombinant polynucleotide as detailed herein, or the vector as detailed herein.

Another aspect of the disclosure provides a kit comprising the system as detailed herein, the recombinant polynucleotide as detailed herein, or the vector as detailed herein, or the composition as detailed herein.

Another aspect of the disclosure provides a method for restoring dystrophin function in a cell or a subject having a mutant dystrophin gene. The method may include contacting the cell or the subject with the system as detailed herein, the recombinant polynucleotide as detailed herein, or the vector as detailed herein, or the composition as detailed herein. In some embodiments, the dystrophin function is restored by insertion of exon 52 of the wild-type dystrophin gene. In some embodiments, the subject is suffering from Duchenne Muscular Dystrophy.

Another aspect of the disclosure provides a method for restoring dystrophin function in a cell or a subject having a disrupted dystrophin gene caused by one or more deleted or mutated exons. The method may include contacting the cell or the subject with the system as detailed herein, the recombinant polynucleotide as detailed herein, or the vector as detailed herein, or the composition as detailed herein. In some embodiments, dystrophin function is restored by inserting one or more wild-type exons of dystrophin gene corresponding to the one or more deleted or mutated exons. In some embodiments, one of the deleted or mutated exons is exon 52.

### BRIEF DESCRIPTION OF THE DRAWINGS

FIG. 1 is a schematic diagram of the exons encoding the dystrophin protein and various interactions in the cell.

FIG. 2 is a schematic diagram of the dystrophin protein.

FIG. 3 is a diagram of the strategy for generating gRNAs targeting hDMD-Intron51, upstream of hDMD-Exon52.

FIG. 4A shows the primer numbers and expected band sizes for the surveyor analysis shown in FIG. 4B. FIG. 4B are gels showing the editing efficiency of gRNAs targeting hDMD-Intron51, upstream of hDMD-Exon52.

FIG. 5A shows the HEK293T SNP results based on primer locations, with gels shown in FIG. 5B.

FIG. 6A are gels showing myoblasts electroporated with plasmids encoding gRNAs redesigned with 19-23 bp spacers, with further results shown in FIG. 6B.

FIG. 7 are gels showing results of HEK293T transfection of gRNA expression plasmids or AAV-HITI donor plasmids.

FIG. 8A are gels showing the nested PCR results to detect HITI-mediated integration of AAV plasmids (AAV-CMV-Cas9 plasmid and an AAV-U6-gRNA-Ex52 plasmid) that were electroporated into primary myoblasts from hDMDΔ52/mdx mice. FIG. 8B are Sanger sequencing results with the expected HITI-mediated insertion.

FIG. 9 is a schematic diagram of the experiments used to confirm in vivo editing, determine the best gRNA/donor sequence combination, and determine the best ratio of AAV-Cas9 to AAV-donor plasmids.

FIG. 10A is a gel showing targeted Ex52 insertion in the genomic DNA of mice with primers downstream of the targeted cut site. FIG. 10B is a gel showing targeted Ex52 insertion in the genomic DNA of mice with primers upstream of the targeted cut site.

FIG. 11 is a gel showing targeted Ex52 insertion in mRNA of treated hDMDΔ52mdx mice.

FIG. 12 is a Western blot analysis confirming protein restoration in treated mice.

FIG. 13 are results from Illumina deep sequencing quantification of AAV-ITR genomic integration in edited mice.

FIG. 14A is a gel showing the amplification of cDNA from exon 45 to exon 69. FIG. 14B is from PacBio sequencing analysis of mRNA, showing that the sequencing reads with 118 bp between exon 51 and exon 53 match the exon 52 sequence.

### DETAILED DESCRIPTION

The present disclosure provides CRISPR/Cas-based gene/genome editing compositions and methods for treating Duchenne Muscular Dystrophy (DMD) by restoring dystrophin function. DMD is typically caused by deletions in the dystrophin gene that disrupt the reading frame. Many strategies to treat DMD aim to restore the reading frame by removing or skipping over an additional exon, as it has been shown that internally truncated dystrophin protein can still be partially functional. Detailed herein are AAV-based Homology-Independent Targeted Integration (HITI)-mediated gene editing therapies for correcting the dystrophin gene. Specifically, we adapted the CRISPR/Cas9 gene editing technology to direct the targeted insertion of missing exons into the dystrophin gene. As a therapeutically relevant target, HITI-mediated genome editing strategies were optimized in a humanized mouse model of DMD in which exon 52 has been removed in mice carrying the full-length human dystrophin gene (hDMDΔ52/mdx mice). To achieve targeted integration, an AAV vector containing the deleted genome sequence including exon 52 was co-delivered with AAV encoding Cas9/gRNA expression cassettes. Targeted exon 52 integration in cultured cells was confirmed. Combined with AAV delivery, HITI-mediated strategies for targeted insertion of missing exons provides a method to restore full-length dystrophin and Unproved functional outcomes.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9. the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

“Binding region” as used herein refers to the region within a target region that is recognized and bound by the CRISPR/Cas-based genome editing system.

“Chromatin” as used herein refers to an organized complex of chromosomal DNA associated with histones.

“Clustered Regularly Interspaced Short Palindromic Repeats” and “CRISPRs”, as used interchangeably herein refers to loci containing multiple short direct repeats that are found in the genomes of approximately 40% of sequenced bacteria and 90% of sequenced archaea.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

“Duchenne Muscular Dystrophy” or “DMD” as used interchangeably herein refers to a recessive, fatal, X-linked disorder that results in muscle degeneration and eventual death. DMD is a common hereditary monogenic disease and occurs in 1 in 3500 males. DMD is the result of inherited or spontaneous mutations that cause nonsense or frame shift mutations in the dystrophin gene. The majority of dystrophin mutations that cause DMD are deletions of exons that disrupt the reading frame and cause premature translation termination in the dystrophin gene. DMD patients typically lose the ability to physically support themselves during childhood, become progressively weaker during the teenage years, and die in their twenties.

“Dystrophin” as used herein refers to a rod-shaped cytoplasmic protein which is a part of a protein complex that connects the cytoskeleton of a muscle fiber to the surrounding extracellular matrix through the cell membrane. Dystrophin provides structural stability to the dystroglycan complex of the cell membrane that is responsible for regulating muscle cell integrity and function. The dystrophin gene or “DMD gene” as used interchangeably herein is 2.2 megabases at locus Xp21. The primary transcription measures about 2,400 kb with the mature mRNA being about 14 kb. 79 exons code for the protein which is over 3500 amino acids.

“Exon 52” as used herein refers to the 52nd exon of the dystrophin gene. Exon 52 is frequently adjacent to frame-disrupting deletions in DMD patients. Exon 52 may comprise the polynucleotide of SEQ ID NO: 21. Exon 52 may be comprised within a polynucleotide of SEQ ID NO: 22.

“Enhancer” as used herein refers to non-coding DNA sequences containing multiple activator and repressor binding sites. Enhancers range from 200 bp to 1 kb in length and may be either proximal, 5′ upstream to the promoter or within the first intron of the regulated gene, or distal, in introns of neighboring genes or intergenic regions far away from the locus. Through DNA looping, active enhancers contact the promoter dependently of the core DNA binding motif promoter specificity. 4 to 5 enhancers may interact with a promoter. Similarly, enhancers may regulate more than one gene without linkage restriction and may “skip” neighboring genes to regulate more distant ones. Transcriptional regulation may involve elements located in a chromosome different to one where the promoter resides. Proximal enhancers or promoters of neighboring genes may serve as platforms to recruit more distal elements.

“Functional” and “full-functional” as used herein describes protein that has biological activity. A “functional gene” refers to a gene transcribed to mRNA, which is translated to a functional protein.

“Fusion protein” as used herein refers to a chimeric protein created through the joining of two or more genes that originally coded for separate proteins. The translation of the fusion gene results in a single polypeptide with functional properties derived from each of the original proteins.

“Genetic construct” as used herein refers to the DNA or RNA molecules that comprise a nucleotide sequence that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operably linked to a coding sequence that encodes a protein such that when present in the cell of the individual, the coding sequence will be expressed.

“Genome editing” as used herein refers to changing a gene. Genome editing may include correcting or restoring a mutant gene. Genome editing may alter a splice acceptor site. Genome editing may be used to treat disease or enhance muscle repair by changing the gene of interest.

The term “heterologous” as used herein refers to nucleic acid comprising two or more subsequences that are not found in the same relationship to each other in nature. For instance, a nucleic acid that is recombinantly produced typically has two or more sequences from unrelated genes synthetically arranged to make a new functional nucleic acid, e.g., a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include a non-native (non-naturally occurring) nucleic acid that has integrated into the chromosome, or a non-native (non-naturally occurring) extrachromosomal nucleic acid. Similarly, a heterologous protein indicates that the protein comprises two or more subsequences that are not found in the same relationship to each other in nature (e.g., a “fusion protein,” where the two subsequences are encoded by a single nucleic acid sequence).

“Identical” or “identity” as used herein in the context of two or more nucleic acids or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” as used herein refers to a mutant gene that has a mutation that causes a premature stop codon. The disrupted gene product is truncated relative to a full-length undisrupted gene product.

“Normal gene” as used herein refers to a gene that has not undergone a change, such as a loss, gain, or exchange of genetic material. The normal gene undergoes normal gene transmission and gene expression. For example, a normal gene may be a wild-type gene.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Open reading frame” refers to a stretch of codons that begins with a start codon and ends at a stop codon. In eukaryotic genes with multiple exons, introns are removed and exons are then joined together after transcription to yield the final mRNA for protein translation. An open reading frame may be a continuous stretch of codons. In some embodiments, the open reading frame only applies to spliced mRNAs, not genomic DNA, for expression of a protein.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain. With respect to fusion polypeptides, the terms “operatively linked” and “operably linked” can refer to the fact that each of the components performs the same function in linkage to the other component as it would if it were not so linked.

“Partially-functional” as used herein describes a protein that is encoded by a mutant gene and has less biological activity than a functional protein but more than a non-functional protein.

“Premature stop codon” or “out-of-frame stop codon” as used interchangeably herein refers to nonsense mutation in a sequence of DNA, which results in a stop codon at location not normally found in the wild-type gene. A premature stop codon may cause a protein to be truncated or shorter compared to the full-length version of the protein.

“Promoter” as used herein means a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter. RSV-LTR promoter. CMV IE promoter, SV40 early promoter or SV40 late promoter and the CMV IE promoter.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed or not expressed at all.

“Skeletal muscle” as used herein refers to a type of striated muscle, which is under the control of the somatic nervous system and attached to bones by bundles of collagen fibers known as tendons. Skeletal muscle is made up of individual components known as myocytes, or “muscle cells”, sometimes colloquially called “muscle fibers.” Myocytes are formed from the fusion of developmental myoblasts (a type of embryonic progenitor cell that gives rise to a muscle cell) in a process known as myogenesis. These long, cylindrical, multinucleated cells are also called myofibers.

“Skeletal muscle condition” as used herein refers to a condition related to the skeletal muscle, such as muscular dystrophies, aging, muscle degeneration, wound healing, and muscle weakness or atrophy.

“Subject” and “patient” as used herein interchangeably refers to any vertebrate, including, but not limited to, a mammal (e.g., cow, pig, camel, llama, hedgehog, anteater, platypus, elephant, alpaca, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some embodiments, the subject may be a human or a non-human. The subject or patient may be undergoing other forms of treatment.

“Treat”, “treating,” or “treatment” are each used interchangeably herein to describe reversing, alleviating, or inhibiting the progress of a disease, or one or more symptoms of such disease, to which such term applies. A treatment may be either performed in an acute or chronic way. The term also refers to reducing the severity of a disease or symptoms associated with such disease prior to affliction with the disease. Such reduction of the severity of a disease prior to affliction refers to administration of an antibody or pharmaceutical composition to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes may be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be a viral vector, bacteriophage, bacterial artificial chromosome or yeast artificial chromosome. A vector may be a DNA or RNA vector. A vector may be a self-replicating extrachromosomal vector, and preferably, is a DNA plasmid. For example, the vector may encode the CRISPR/Cas-based genome editing system described herein, including a polynucleotide sequence encoding a Cas protein or fusion protein, and/or at least one gRNA nucleotide sequence of SEQ ID NO: 19 or SEQ ID NO: 20 or a gRNA targeting a nucleotide sequence comprising SEQ ID NO: 17 or SEQ ID NO: 18.

### 2. CRISPR/Cas-BASED GENOME EDITING SYSTEM FOR RESTORING DYSTROPHIN

Provided herein are CRISPR/Cas-based genome editing systems for use in restoring dystrophin gene function. In some embodiments, the CRISPR/Cas-based genome editing system includes a Cas protein or fusion protein and at least one guide RNA (gRNA) that binds and targets a polynucleotide sequence corresponding to SEQ ID NO: 17 or SEQ ID NO: 18. In some embodiments, the at least one guide RNA (gRNA) comprises or is encoded by a polynucleotide of SEQ ID NO: 19 or SEQ ID NO: 20. The fusion protein can comprise two heterologous polypeptide domains. In some embodiments, the fusion protein comprises a Cas protein and a base-editing domain, or a domain with other enzymatic function. In some embodiments, the at least one gRNA binds and targets a polynucleotide sequence corresponding to: a) a fragment of SEQ ID NO: 17 or SEQ ID NO: 18; b) a complement of SEQ ID NO: 17 or SEQ ID NO: 18, or fragment thereof; c) a nucleic acid that is substantially identical to SEQ ID NO: 17 or SEQ ID NO: 18, or complement thereof; or d) a nucleic acid that hybridizes under stringent conditions to SEQ ID NO: 17 or SEQ ID NO: 18, complement thereof, or a sequence substantially identical thereto.

### a) Dystrophin Gene

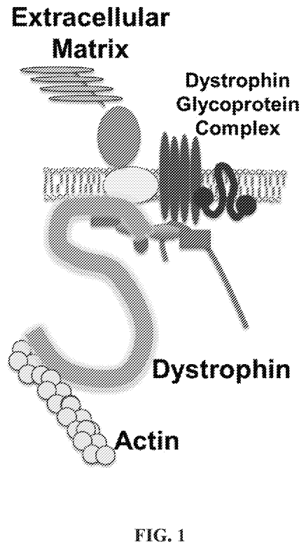

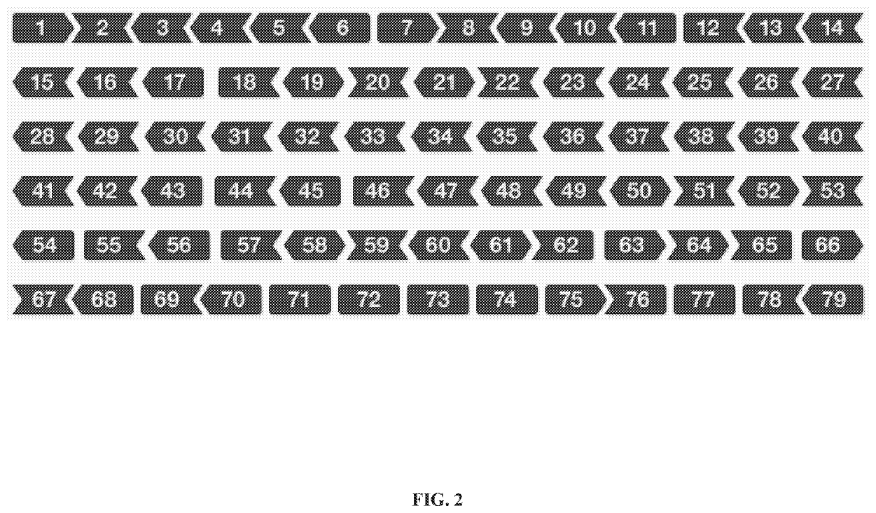

Dystrophin is a rod-shaped cytoplasmic protein which is a part of a protein complex that connects the cytoskeleton of a muscle fiber to the surrounding extracellular matrix through the cell membrane (FIG. 1). Dystrophin provides structural stability to the dystroglycan complex of the cell membrane. The dystrophin gene is 2.2 megabases at locus Xp21. The primary transcription measures about 2,400 kb with the mature mRNA being about 14 kb. 79 exons include approximately 2.2 million nucleotides and code for the protein which is over 3500 amino acids (FIG. 2). Normal skeleton muscle tissue contains only small amounts of dystrophin but its absence of abnormal expression leads to the development of severe and incurable symptoms. Some mutations in the dystrophin gene lead to the production of defective dystrophin and severe dystrophic phenotype in affected patients. Some mutations in the dystrophin gene lead to partially-functional dystrophin protein and a much milder dystrophic phenotype in affected patients.

DMD is the result of inherited or spontaneous mutations that cause nonsense or frame shift mutations in the dystrophin gene. DMD is the most prevalent lethal heritable childhood disease and affects approximately one in 5,000 newborn males. DMD is characterized by progressive muscle weakness, often leading to mortality in subjects at age mid-twenties, due to the lack of a functional dystrophin gene. Most mutations are deletions in the dystrophin gene that disrupt the reading frame. Naturally occurring mutations and their consequences are relatively well understood for DMD. It is known that in-frame deletions that occur in the exon 45-55 regions contained within the rod domain can produce highly functional dystrophin proteins, and many carriers are asymptomatic or display mild symptoms. Exons 45-55 of dystrophin are a mutational hotspot. Furthermore, more than 60% of patients may theoretically be treated by targeting exons in this region of the dystrophin gene. Efforts have been made to restore the disrupted dystrophin reading frame in DMD patients by skipping non-essential exon(s) (e.g., exon 45 skipping) during mRNA splicing to produce internally deleted but functional dystrophin proteins. The deletion of internal dystrophin exon(s) (e.g., deletion of exon 45) retains the proper reading frame and can generate an internally truncated but partially functional dystrophin protein. Deletions between exons 45-55 of dystrophin result in a phenotype that is much milder compared to DMD.

A dystrophin gene may be a mutant dystrophine gene. A dystrophin gene may be a wild-type dystrophine gene. A dystrophin gene may have a sequence that is functionally identical to a wild-type dystrophine gene, for example, the sequence may be codon-optimized but still encode for the same protein as the wild-type dystrophin. A mutant dystrophin gene may include one or more mutations relative to the wild-type dystrophin gene. Mutations may include, for example, nucleotide deletions, substitutions, additions, transversions, or combinations thereof. Mutations may include deletions of all or parts of at least one intron and/or exon. An exon of a mutant dystrophin gene may be mutated or at least partially deleted from the dystrophin gene. An exon of a mutant dystrophin gene may be fully deleted. A mutant dystrophin gene may have a portion or fragment thereof that corresponds to the corresponding sequence in the wild-type dystrophin gene. In some embodiments, a disrupted dystrophin gene caused by a deleted or mutated exon can be restored in DMD patients by adding back the corresponding wild-type exon. In some embodiments, disrupted dystrophin caused by a deleted or mutated exon 52 can be restored in DMD patients by adding back in wild-type exon 52. In certain embodiments, addition of exon 52 to restore reading frame ameliorates the phenotype in DMD subjects, including DMD subjects with deletion mutations. In certain embodiments, one or more exons may be added and inserted into the disrupted dystrophin gene. The one or more exons may be added and inserted so as to restore the corresponding mutated or deleted exon(s) in dystrophin. The one or more exons may be added and inserted into the disrupted dystrophin gene in addition to adding back and inserting the exon 52. In certain embodiments, exon 52 of a dystrophin gene refers to the 52nd exon of the dystrophin gene. Exon 52 is frequently adjacent to frame-disrupting deletions in DMD patients and has been targeted in clinical trials for oligonucleotide-based exon skipping.

A presently disclosed genetic construct (e.g., a vector) can mediate highly efficient exon 52 addition into a dystrophin gene (e.g., the human dystrophin gene). A presently disclosed genetic construct (e.g., a vector) can restore dystrophin protein expression in cells from DMD patients. Exon 52 is frequently adjacent to frame-disrupting deletions in DMD. Addition of exon 52 to the dystrophin transcript can be used to treat DMD patients. A presently disclosed genetic construct (e.g., a vector) may be transfected into human DMD cells and mediate efficient gene modification and conversion to the correct reading frame. Protein restoration may be concomitant with frame restoration and detected in a bulk population of CRISPR/Cas-based genome editing system-treated cells.

### b) Fusion Protein

The CRISPR/Cas-based gene editing system may include a fusion protein, or a nucleic acid sequence encoding a fusion protein. The fusion protein may include a Cas protein and a gene/genome-editing domain, or a domain with other enzymatic function. In some embodiments, the nucleic acid sequence encoding the fusion protein is DNA. In some embodiments, the nucleic acid sequence encoding the fusion protein is RNA.

### i) Cas Protein

The CRISPR/Cas-based gene editing system may include a Cas protein. The Cas protein forms a complex with the 3′ end of a gRNA. The specificity of the CRISPR-based system depends on two factors: the target sequence and the protospacer-adjacent motif (PAM). The target sequence is located on the 5′ end of the gRNA and is designed to bond with base pairs on the host DNA at the correct DNA sequence known as the protospacer. By simply exchanging the recognition sequence of the gRNA, the Cas protein can be directed to new genomic targets. The PAM sequence is located on the DNA to be altered and is recognized by a Cas protein. PAM recognition sequences of the Cas protein can be species specific.

Cas9 protein is an endonuclease that cleaves nucleic acid and is encoded by the CRISPR loci and is involved in the Type II CRISPR system. A Cas9 molecule can interact with one or more gRNA molecules and, in concert with the gRNA molecule(s), localizes to a site which comprises a target domain, and in certain embodiments, a PAM sequence. The ability of a Cas9 molecule to recognize a PAM sequence can be determined, e.g., using a transformation assay as known in the art. In some embodiments, the CRISPR/Cas-based gene editing system includes a Cas9 protein from Streptococcus pyogenes In some embodiments, the Cas9 protein comprises the amino acid sequence of SEQ ID NO: 1. In some embodiments, the CRISPR/Cas-based gene editing system includes a Cas9 protein from Staphylococcus aureus. In some embodiments, the Cas9 protein comprises the amino acid sequence of SEQ ID NO: 2.

In some embodiments, the CRISPR/Cas-based gene editing system includes a catalytically dead dCas9. In some embodiments, the Cas9 protein may be mutated so that the nuclease activity is inactivated. An inactivated Cas9 protein (“iCas9”, also referred to as “dCas9”) with no endonuclease activity may be targeted to genes in bacteria, yeast, and human cells by gRNAs to silence gene expression through steric hindrance. Exemplary mutations with reference to the S. pyogenes Cas9 sequence to inactivate the nuclease activity include: D10A, E762A, H840A, N854A, N863A and/or D986A. A S. pyogenes Cas9 protein with the D10A mutation may comprise an amino acid sequence of SEQ ID NO: 3. A S. pyogenes Cas9 protein with D10A and H849A mutations may comprise an amino acid sequence of SEQ ID NO: 4. Exemplary mutations with reference to the S. aureus Cas9 sequence to inactivate the nuclease activity include D10A and N580A.

The Cas9 protein or mutant Cas9 protein may be from any bacterial or archaea species, such as Streptococcus pyogenes, Staphylococcus aureus, Streptococcus thermophiles, or Neisseria meningitides. In some embodiments, the Cas protein or mutant Cas9 protein is a Cas9 protein derived from a bacterial genus of Streptococcus, Staphylococcus, Brevibacillus, Corynebacter, Sutterella, Legionella, Francisella, Treponema, Filifactor, Eubacterium, Lactobacillus, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, or Campylobacter. In some embodiments, the Cas9 protein or mutant Cas9 protein is selected from the group including, but not limited to, Streptococcus pyogenes, Francisella novicida, Staphylococcus aureus, Neisseria meningitides, Streptococcus thermophiles, Treponema denticola, Brevibacillus laterosporus, Campylobacter jejuni, Corynebacterium diphtheria, Eubacterium ventriosum, Streptococcus pasteurianus, Lactobacillus farciminis, Sphaerochaeta globus, Azospirillum, Gluconacetobacter diazotrophicus, Neisseria cinerea, Roseburia intestinalis, Parvibaculum lavamentivorans, Nitratifractor salsuginis, and Campylobacter lari.

In certain embodiments, the ability of a Cas9 molecule or mutant Cas9 protein to interact with and cleave a target nucleic acid is PAM sequence dependent. A PAM sequence is a sequence in the target nucleic acid. In certain embodiments, cleavage of the target nucleic acid occurs upstream from the PAM sequence. Cas9 molecules from different bacterial species can recognize different sequence motifs (e.g., PAM sequences). In certain embodiments, a Cas9 molecule of S. pyogenes recognizes the sequence motif NGG (SEQ ID NO: 10) and directs cleavage of a target nucleic acid sequence 1 to 10, such as 3 to 5, bp upstream from that sequence (see, e.g., Mali 2013). In certain embodiments, a Cas9 molecule of S. aureus recognizes the sequence motif NNGRR (R=A or G) (SEQ ID NO: 12) and directs cleavage of a target nucleic acid sequence 1 to 10, such as 3 to 5, bp upstream from that sequence. In certain embodiments, a Cas9 molecule of S. aureus recognizes the sequence motif NNGRRN (R=A or G) (SEQ ID NO: 13) and directs cleavage of a target nucleic acid sequence 1 to 10, such as 3 to 5, bp upstream from that sequence. In certain embodiments, a Cas9 molecule of S. aureus recognizes the sequence motif NNGRRT (R=A or G) (SEQ ID NO: 14) and directs cleavage of a target nucleic acid sequence 1 to 10, such as 3 to 5, by upstream from that sequence. In certain embodiments, a Cas9 molecule of S. aureus recognizes the sequence motif NNGRRV (R=A or G; V=A or C or G) (SEQ ID NO: 15) and directs cleavage of a target nucleic acid sequence 1 to 10, such as 3 to 5, bp upstream from that sequence. In the aforementioned embodiments, N can be any nucleotide residue, e.g., any of A, G, C, or T. Cas9 molecules can be engineered to alter the PAM specificity of the Cas9 molecule.

In some embodiments, the Cas9 protein or mutant Cas9 protein can recognize a PAM sequence NGG (SEQ ID NO: 10) or NGA (SEQ ID NO: 16). In some embodiments, the Cas9 protein or mutant Cas9 protein can recognize a PAM sequence NNNRRT (SEQ ID NO: 11). In some embodiments, the Cas9 protein or mutant Cas9 protein can recognize a PAM sequence ATTCCT (SEQ ID NO: 9). In some embodiments, the Cas9 protein or mutant Cas9 protein is a Cas9 protein of S. aureus and recognizes the sequence motif NNGRR (R=A or G) (SEQ ID NO: 12), NNGRRN (R=A or G) (SEQ ID NO: 13), NNGRRT (R=A or G) (SEQ ID NO: 14), or NNGRRV (R=A or G) (SEQ ID NO: 15). In the aforementioned embodiments, N can be any nucleotide residue, e.g., any of A, G, C, or T. Cas9 molecules can be engineered to alter the PAM specificity of the Cas9 molecule.

Additionally or alternatively, a nucleic acid encoding a Cas9 molecule or Cas9 polypeptide may comprise a nuclear localization sequence (NLS). Nuclear localization sequences are known in the art.

### c) gRNA

The CRISPR/Cas-based genome editing system may include at least one gRNA. The gRNA may target a fragment of a dystrophin gene. The gRNA may target a fragment of a mutant dystrophin gene. The gRNA may target a fragment of a wild-type dystrophin gene. A fragment may be about 5 to about 200, about 10 to about 200, about 5 to about 300, or about 10 to about 300 nucleotides in length. A fragment may be at least about 5, at least about 10, at least about 15, at least about 20, at least about 30, at least about 40, at least about 50, or at least about 100 nucleotides in length. gRNA may target a fragment or portion of the dystrophin gene that comprises a mutation or deletion, or a sequence proximal or juxtapositioned thereto. The gRNA may target an intron that is juxtaposed with an exon of the dystrophin gene. The gRNA may target an intron that is juxtaposed with an exon of a mutant dystrophin gene. The fragment of a wild-type dystrophin gene may be flanked by two gRNA spacers and/or PAM sequences, as detailed herein. Each gRNA spacer may comprise an amino acid sequence selected from SEQ ID NOs: 5-8 and 25-45. The two gRNA spacers may be identical. The two gRNA spacers may be different. In some embodiments, at least one of the two gRNA spacers comprises a sequence of SEQ ID NO: 25 or SEQ ID NO: 26. The exon may be selected from exons 1-8, 10, 11, 12, 14, 16-22, 43-59, and 61-66 of the dystrophin gene. In some embodiments, the exon is exon 52. The gRNA provides the targeting of the CRISPR/Cas-based gene editing systems. The gRNA is a fusion of two noncoding RNAs: a crRNA and a tracrRNA. The sgRNA may target any desired DNA sequence by exchanging the sequence encoding a 20 bp protospacer which confers targeting specificity through complementary base pairing with the desired DNA target. gRNA mimics the naturally occurring crRNA:tracrRNA duplex involved in the Type II Effector system. This duplex, which may include, for example, a 42-nucleotide crRNA and a 75-nucleotide tracrRNA, acts as a guide for the Cas9.

In some embodiments, at least one gRNA may target and bind a target region. In some embodiments, between 1 and 20 gRNAs may be used to alter a target gene, for example, to alter a splice acceptor site. For example, between 1 gRNA and 20 gRNAs, between 1 gRNA and 15 gRNAs, between 1 gRNA and 10 gRNAs, between 1 gRNA and 5 gRNAs, between 2 gRNAs and 20 gRNAs, between 2 gRNAs and 15 gRNAs, between 2 gRNAs and 10 gRNAs, between 2 gRNAs and 5 gRNAs, between 5 gRNAs and 20 gRNAs, between 5 gRNAs and 15 gRNAs, or between 5 gRNAs and 10 gRNAs may be included in the CRISPR/Cas-based gene editing system and used to alter the splice acceptor site. In some embodiments, at least 1 gRNA, at least 2 gRNAs, at least 3 gRNAs, at least 4 gRNAs, at least 5 gRNAs, at least 6 gRNAs, at least 7 gRNAs, at least 8 gRNAs, at least 9 gRNAs, at least 10 gRNAs, at least 11 gRNAs, at least 12 gRNAs, at least 13 gRNAs, at least 14 gRNAs, at least 15 gRNAs, or at least 20 gRNAs may be included in the CRISPR/Cas-based gene editing system and used to alter the splice acceptor site. In some embodiments, less than 30 gRNAs, less than 25 gRNAs, less than 20 gRNAs, less than 15 gRNAs, less than 10 gRNAs, less than 5 gRNAs, or less than 3 gRNAs may be included in the CRISPR/Cas-based gene editing system and used to alter the splice acceptor site.

The CRISPR/Cas-based gene editing system may use gRNA of varying sequences and lengths. The gRNA may comprise a complementary polynucleotide sequence of the target DNA sequence, such as a target sequence comprising SEQ ID NO: 17 or SEQ ID NO: 18 or a complementary polynucleotide sequence of a target sequence comprising SEQ ID NO: 17 or SEQ ID NO: 18, followed by NGG. The gRNA may comprise a “G” at the 5′ end of the complementary polynucleotide sequence. The gRNA may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by NGG. The gRNA may comprise less than a 40 base pair, less than a 35 base pair, less than a 30 base pair, less than a 25 base pair, less than a 20 base pair, or less than a 15 base pair complementary polynucleotide sequence of the target DNA sequence followed by NGG. The gRNA may target at least one of the promoter region, the enhancer region or the transcribed region of the target gene.

The at least one gRNA may bind and target a nucleic acid sequence comprising SEQ ID NO: 17 or SEQ ID NO: 18. The target sequence may comprise a polynucleotide of SEQ ID NO: 17 or SEQ ID NO 18, or a fragment thereof, or a truncation thereof, such as a 5′-truncation. A truncation may be 1, 2, 3, 4, 5, 6, 7, 8, or 9 nucleotides shorter than the sequence of SEQ ID NO: 17 or SEQ ID NO: 18. The gRNA may comprise a polynucleotide corresponding to SEQ ID NO: 17 or SEQ ID NO: 18, a complement thereof, a variant thereof, or fragment thereof. The gRNA may be encoded by a polynucleotide sequence comprising SEQ ID NO: 17 or SEQ ID NO: 18. The portion of the gRNA that targets the target sequence in the genome may be referred to as a gRNA spacer or a protospacer. The protospacer may be defined as the portion of the gRNA that is complementary to the targeted sequence in the genome. The protospacer may comprise a polynucleotide of SEQ ID NO: 17 or SEQ ID NO 18, or a fragment thereof, or a truncation thereof, or a complement thereof. The gRNA may include a gRNA scaffold. A gRNA scaffold facilitates Cas9 binding to the gRNA and endonuclease activity. The gRNA scaffold is a polynucleotide sequence that follows the portion of the gRNA corresponding to sequence that the gRNA targets. Together, the gRNA targeting portion and gRNA scaffold form one polynucleotide. In some embodiments, the gRNA targeting portion and gRNA scaffold together may comprise the polynucleotide sequence of SEQ ID NO: 19 or SEQ ID NO: 20, or a complement thereof. In some embodiments, the gRNA targeting portion and gRNA scaffold together is encoded by the polynucleotide sequence of SEQ ID NO: 19 or SEQ ID NO: 20, or a complement thereof. The gRNA may be encoded by the polynucleotide of SEQ ID NO: 19, a complement thereof, a variant thereof or fragment thereof, or of SEQ ID NO: 20, a complement thereof, a variant thereof, or fragment thereof.

### d) Donor Sequence

The CRISPR/Cas-based gene editing system may include at least one donor sequence. A donor sequence may comprise a fragment of the dystrophin gene. For example, a donor sequence may comprise a nucleic acid sequence encoding an exon or any combination of exons of the dystrophin gene. The donor sequence may comprise an exon of the wild-type dystrophin gene or a functional equivalent thereof. The exon may be selected from exons 1-8, 10, 11, 12, 14, 16-22, 43-59, 61-66 of the dystrophin gene. In some embodiments, the exon is exon 52 of the dystrophin gene. The donor sequence may include a fragment of a wild-type dystrophin gene or a functional equivalent thereof, and the fragment or functional equivalent thereof may be flanked by two gRNA spacers. The donor sequence may further include at least one additional polynucleotide corresponding to intron sequences surrounding or near the exon(s) to be inserted. The donor sequence may further include at least one additional polynucleotide corresponding to intron sequences surrounding or near exon 52. The donor sequence may include a nucleic acid sequence of at least one of SEQ ID NO: 21 or SEQ ID NO: 22, a complement thereof, a variant thereof, or fragment thereof.

The gRNA and donor sequence may be present in a variety of molar ratios. The molar ratio between the gRNA and donor sequence may be 1:1, or 1:15, or from 5:1 to 1:10, or from 1:1 to 1:5. The molar ratio between the gRNA and donor sequence may be at least 1:1, at least 1:2, at least 1:3, at least 1:4, at least 1:5, at least 1:6, at least 1:7, at least 1:8, at least 1:9, at least 1:10, at least 1:15, or at least 1:20. The molar ratio between the gRNA and donor sequence may be less than 20:1, less than 15:1, less than 10:1, less than 9:1, less than 8:1, less than 7:1, less than 6:1, less than 5:1, less than 4:1, less than 3:1, less than 2:1, or less than 1:1.

### 3. COMPOSITIONS FOR RESTORING DYSTROPHIN FUNCTION

Disclosed herein are compositions for restoring dystrophin function. The compositions may restore dystrophin function by adding one or more exons to restore the reading frame of dystrophin. For example, an exon to be added may be exon 52. The composition may include the CRISPR/Cas-based gene editing system, as disclosed above. The composition may also include a viral delivery system. For example, the viral delivery system may include an adeno-associated virus vector or a modified lentiviral vector.

Methods of introducing a nucleic acid into a host cell are known in the art, and any known method can be used to introduce a nucleic acid (e.g., an expression construct) into a cell. Suitable methods include, for example, viral or bacteriophage infection, transfection, conjugation, protoplast fusion, polycation or lipid:nucleic acid conjugates, lipofection, electroporation, nucleofection, immunoliposomes, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. In some embodiments, the composition may be delivered by mRNA delivery and ribonucleoprotein (RNP) complex delivery.

### a) Constructs and Plasmids

The compositions or systems, as described above, may comprise genetic constructs that encodes the CRISPR/Cas-based gene editing system, as disclosed herein. The genetic construct, such as a plasmid or expression vector, may comprise a nucleic acid that encodes the CRISPR/Cas-based gene editing system and/or at least one of the gRNAs. The compositions, as described above, may comprise genetic constructs that encodes the modified Adeno-associated virus (AAV) vector and a nucleic acid sequence that encodes the CRISPR/Cas-based gene editing system, as disclosed herein. In some embodiments, the compositions, as described above, may comprise genetic constructs that encodes the modified adenovirus vector and a nucleic acid sequence that encodes the CRISPR/Cas-based gene editing system, as disclosed herein. The genetic construct, such as a plasmid, may comprise a nucleic acid that encodes the CRISPR/Cas-based gene editing system. The compositions, as described above, may comprise genetic constructs that encodes a modified lentiviral vector. The genetic construct, such as a plasmid, may comprise a nucleic acid that encodes the Cas protein or fusion protein and the at least one gRNA. The genetic construct may be present in the cell as a functioning extrachromosomal molecule. The genetic construct may be a linear minichromosome including centromere, telomeres or plasmids or cosmids.

The genetic construct may also be part of a genome of a recombinant viral vector, including recombinant lentivirus, recombinant adenovirus, and recombinant adenovirus associated virus. The genetic construct may be part of the genetic material in attenuated live microorganisms or recombinant microbial vectors which live in cells. The genetic constructs may comprise regulatory elements for gene expression of the coding sequences of the nucleic acid. The regulatory elements may be a promoter, an enhancer, an initiation codon, a stop codon, or a polyadenylation signal.

The nucleic acid sequences may make up a genetic construct that may be a vector. The vector may be capable of expressing the Cas protein or fusion protein, such as the CRISPR/Cas-based gene editing system, in the cell of a mammal. The vector may be recombinant. The vector may comprise heterologous nucleic acid encoding the Cas protein or fusion protein, such as the CRISPR/Cas-based gene editing system. The vector may be a plasmid. The vector may be useful for transfecting cells with nucleic acid encoding the CRISPR/Cas-based gene editing system, which the transformed host cell is cultured and maintained under conditions wherein expression of the CRISPR/Cas-based gene editing system takes place.

Coding sequences may be optimized for stability and high levels of expression. In some instances, codons are selected to reduce secondary structure formation of the RNA such as that formed due to intramolecular bonding.

The vector may comprise heterologous nucleic acid encoding the CRISPR/Cas-based gene editing system and may further comprise an initiation codon, which may be upstream of the CRISPR/Cas-based gene editing system coding sequence, and a stop codon, which may be downstream of the CRISPR/Cas-based gene editing system coding sequence. The initiation and termination codon may be in frame with the CRISPR/Cas-based gene editing system coding sequence. The vector may also comprise a promoter that is operably linked to the CRISPR/Cas-based gene editing system coding sequence. The promoter may be a ubiquitous promoter. The promoter may be a tissue-specific promoter. The tissue specific promoter may be a muscle specific promoter. The CRISPR/Cas-based gene editing system may be under the light-inducible or chemically inducible control to enable the dynamic control of gene/genome editing in space and time. The promoter operably linked to the CRISPR/Cas-based gene editing system coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metalothionein. The promoter may also be a tissue specific promoter, such as a muscle or skin specific promoter, natural or synthetic. Examples of such promoters are described in US Patent Application Publication No. US20040175727, the contents of which are incorporated herein in its entirety. The promoter may be a CK8 promoter, a Spc512 promoter, a MHCK7 promoter, for example.

The vector may also comprise a polyadenylation signal, which may be downstream of the CRISPR/Cas-based gene editing system. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 vector (Invitrogen, San Diego. Calif.),

The vector may also comprise an enhancer upstream of the CRISPR/Cas-based gene editing system or sgRNAs. The enhancer may be necessary for DNA expression. The enhancer may be human actin, human myosin, human hemoglobin, human muscle creatine or a viral enhancer such as one from CMV, HA, RSV, or EBV. Polynucleotide function enhancers are described in U.S. Pat. Nos. 5,593,972, 5,962,428, and WO94/016737, the contents of each are fully incorporated by reference. The vector may also comprise a mammalian origin of replication in order to maintain the vector extrachromosomally and produce multiple copies of the vector in a cell. The vector may also comprise a regulatory sequence, which may be well suited for gene expression in a mammalian or human cell into which the vector is administered. The vector may also comprise a reporter gene, such as green fluorescent protein (“GFP”) and/or a selectable marker, such as hygromycin (“Hygro”).

The vector may be expression vectors or systems to produce protein by routine techniques and readily available starting materials including Sambrook et al., Molecular Cloning and Laboratory Manual, Second Ed., Cold Spring Harbor (1989), which is incorporated fully by reference. In some embodiments the vector may comprise the nucleic acid sequence encoding the CRISPR/Cas-based gene editing system, including the nucleic acid sequence encoding the Cas protein or fusion protein and the nucleic acid sequence encoding the at least one gRNA comprising the nucleic acid sequence of SEQ ID NO: 19, a complement thereof, a variant thereof, or a fragment thereof, or of SEQ ID NO: 20, a complement thereof, a variant thereof, or a fragment thereof, or a gRNA targeting the nucleic acid sequence of SEQ ID NO: 17 or SEQ ID NO: 18, a variant thereof, or a fragment thereof. In some embodiments two vectors may comprise the nucleic acid sequence encoding the CRISPR/Cas-based gene editing system, including a first vector comprising the nucleic acid sequence encoding the Cas protein or fusion protein and a second vector comprising the nucleic acid sequence encoding the at least one gRNA.

In some embodiments, the compositions are delivered by mRNA and protein/RNA complexes (Ribonucleoprotein (RNP)). For example, the purified Cas protein or fusion protein can be combined with guide RNA to form air RNP complex. The herein described methods may also require the deliver of a DNA donor sequence as described herein.

### b) Modified Lentiviral Vector

The compositions for adding or inserting exon 52 may include a modified lentiviral vector. The modified lentiviral vector includes a first polynucleotide sequence encoding a Cas protein or fusion protein and a second polynucleotide sequence encoding the at least one gRNA. The first polynucleotide sequence may be operably linked to a promoter. The promoter may be a constitutive promoter, an inducible promoter, a repressible promoter, or a regulatable promoter.

The second polynucleotide sequence encodes at least 1 gRNA. For example, the second polynucleotide sequence may encode at least 1 gRNA, at least 2 gRNAs, at least 3 gRNAs, at least 4 gRNAs, at least 5 gRNAs, at least 6 gRNAs, at least 7 gRNAs, at least 8 gRNAs, at least 9 gRNAs, at least 10 gRNAs, at least 11 gRNA, at least 12 gRNAs, at least 13 gRNAs, at least 14 gRNAs, at least 15 gRNAs, at least 16 gRNAs, at least 17 gRNAs, at least 18 gRNAs, at least 19 gRNAs, or at least 20 gRNAs. For example, the second polynucleotide sequence may encode less than 30 gRNAs, less than 25 gRNAs, less than 20 gRNAs, less than 15 gRNAs, less than 10 gRNAs, less than 5 gRNAs, or less than 3 gRNAs. The second polynucleotide sequence may be operably linked to a promoter. The promoter may be a constitutive promoter, an inducible promoter, a repressible promoter, or a regulatable promoter. At least one gRNA may bind to a target gene or loci, such as a target region corresponding to exon 52.

### c) Adeno-Associated Virus Vectors

AAV may be used to deliver the compositions to the cell using various construct configurations. For example, AAV may deliver the Cas protein or fusion protein and the gRNA expression cassettes on separate vectors. Alternatively, both the Cas protein or fusion protein and up to two gRNA expression cassettes may be combined in a single AAV vector within the 4.7 kb packaging limit.

The composition, as described above, includes a modified adeno-associated virus (AAV) vector. The modified AAV vector may be capable of delivering and expressing the site-specific nuclease in the cell of a mammal. For example, the modified AAV vector may be an AAV-SASTG vector (Piacentino et al. (2012) Human Gene Therapy 23:635-646). The modified AAV vector may be based on one or more of several capsid types, including AAV1, AAV2, AAV5, AAV6, AAV8, and AAV9. The modified AAV vector may be based on AAV2 pseudotype with alternative muscle-tropic AAV capsids, such as AAV2/1, AAV2/6, AAV2/7, AAV2/8, AAV2/9, AAV2.5, and AAV/SASTG vectors that efficiently transduce skeletal muscle or cardiac muscle by systemic and local delivery (Seto et al. Current Gene Therapy (2012) 12:139-151). The construct may comprise a polynucleotide sequence of SEQ ID NO: 23. The construct may comprise a polynucleotide sequence of SEQ ID NO: 24.

### 4. METHODS OF RESTORING DYSTROPHIN FUNCTION IN A SUBJECT HAVING A MUTANT DYSTROPHIN GENE

The presently disclosed subject matter provides for methods of restoring dystrophin function (e.g., a mutant dystrophin gene, e.g., a mutant human dystrophin gene) in a cell and/or a subject suffering from DMD and/or having a mutant dystrophin gene. The method can include administering to a cell or subject a CRISPR/Cas-based gene editing system, a polynucleotide or vector encoding said CRISPR/Cas-based gene editing system, or composition of said CRISPR/Cas9-based gene editing system as described above. In some embodiments, the subject is suffering from Duchenne Muscular Dystrophy. In some embodiments, dystrophin function is restored by inserting one or more wild-type exons of dystrophin gene corresponding to the one or more deleted or mutated exons. In some embodiments, the dystrophin function is restored by insertion of exon 52 of the wild-type dystrophin gene.

The method can include administering to a cell or a subject a presently disclosed genetic construct (e.g., a vector) or a composition comprising thereof as described above. The method can comprise administering to the skeletal muscle and/or cardiac muscle of the subject the presently disclosed genetic construct (e.g., a vector) or a composition comprising thereof for genome editing in skeletal muscle and/or cardiac muscle, as described above. Use of presently disclosed genetic construct (e.g., a vector) or a composition comprising thereof to deliver the CRISPR/Cas-based gene editing system to the skeletal muscle or cardiac muscle may restore the expression of a full-functional or partially-functional protein. The CRISPR/Cas-based gene editing system has the advantage of advanced genome editing due to their high rate of successful and efficient genetic modification.

The method may include administering a CRISPR/Cas-based gene editing system, such as administering a Cas protein or fusion protein, a nucleotide sequence encoding said Cas protein or fusion protein, and/or at least one gRNA comprising or encoded by or corresponding to SEQ ID NO: 19, a complement thereof, a variant thereof, or fragment thereof, or comprising or encoded by or corresponding to SEQ ID NO: 20, a complement thereof, a variant thereof, or a fragment thereof, or a gRNA targeting the nucleic acid sequence of SEQ ID NO: 17 or SEQ ID NO: 18, a variant thereof, or a fragment thereof.

Use of presently disclosed genetic construct (e.g., a vector) or a composition comprising thereof to deliver the CRISPR/Cas-based gene editing system to the target muscle, for example, may restore the expression of a full-functional or partially-functional protein with a repair template or donor DNA, which can replace the entire gene or the region containing the mutation. The CRISPR/Cas-based gene editing system may be used to introduce site-specific double strand breaks at targeted genomic loci. Site-specific double-strand breaks are created when the CRISPR/Cas-based gene editing system binds to a target DNA sequences, thereby permitting cleavage of the target DNA. This DNA cleavage may stimulate the natural DNA-repair machinery, which may lead to one of two possible repair pathways: homology-directed repair (HDR) or the non-homologous end joining (NHEJ) pathway, for example.

The disclosed CRISPR/Cas-based gene editing systems may involve using homology-directed repair or nuclease-mediated non-homologous end joining (NHEJ)-based correction approaches, which enable efficient correction in proliferation-limited primary cell lines that may not be amenable to homologous recombination or selection-based gene correction. This strategy integrates the rapid and robust assembly of active CRISPR/Cas-based gene editing systems with an efficient gene editing method for the treatment of genetic diseases caused by mutations in nonessential coding regions that cause frameshifts, premature stop codons, aberrant splice donor sites or aberrant splice acceptor sites.

Restoration of protein expression from an endogenous mutated gene may be through template-free NHEJ-mediated DNA repair. In contrast to a transient method targeting the target gene RNA, the correction of the target gene reading frame in the genome by a transiently expressed CRISPR/Cas-based gene editing system may lead to permanently restored target gene expression by each modified cell and all of its progeny. In certain embodiments, NHEJ is a nuclease mediated NHEJ, which in certain embodiments, refers to NHEJ that is initiated a Cas molecule, cuts double stranded DNA. The method comprises administering a presently disclosed genetic construct (e.g., a vector) or a composition comprising thereof to the skeletal muscle or cardiac muscle of the subject for genome editing in skeletal muscle or cardiac muscle.

Nuclease mediated NHEJ gene correction may correct the mutated target gene and offers several potential advantages over the HDR pathway. For example, NHEJ does not require a donor template, which may cause nonspecific insertional mutagenesis. In contrast to HDR, NHEJ operates efficiently in all stages of the cell cycle and therefore may be effectively exploited in both cycling and post-mitotic cells, such as muscle fibers. This provides a robust, permanent gene restoration alternative to oligonucleotide-based exon skipping or pharmacologic forced read-through of stop codons and could theoretically require as few as one drug treatment. NHEJ-based gene correction using a CRISPR/Cas-based gene editing system, as well as other engineered nucleases including meganucleases and zinc finger nucleases, may be combined with other existing ex vivo and in vivo platforms for cell- and gene-based therapies, in addition to the plasmid electroporation approach described here. For example, delivery of a CRISPR/Cas-based gene editing system by mRNA-based gene transfer or as purified cell permeable proteins could enable a DNA-free genome editing approach that would circumvent any possibility of insertional mutagenesis.

Recently, AAV delivery of CRISPR/Cas9 strategies for homology-independent targeted integration (HITI) has resulted in genome editing of neurons in vivo. See Suzuki, K., Tsunekawa, Y., Hernandez-Benitez, R., et al. In vivo genome editing via CRISPR/Cas9 mediated homology-independent targeted integration. Nature 540, 144-149 (2016). Herein described are AAV-based HITI-mediated gene editing therapies for correcting DMD. Such AAV CRISPR/Cas9 delivery systems may be used to provide efficient and functional correction in humanized animal models of DMD, for example.

### 5. PHARMACEUTICAL COMPOSITIONS

The CRISPR/Cas-based gene editing system may be in a pharmaceutical composition. The pharmaceutical composition may comprise about 1 ng to about 10 mg of DNA encoding the CRISPR/Cas-based gene editing system. The pharmaceutical compositions as detailed herein are formulated according to the mode of administration to be used. In cases where pharmaceutical compositions are injectable pharmaceutical compositions, they are sterile, pyrogen free and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The pharmaceutical composition containing the CRISPR/Cas-based gene editing system may further comprise a pharmaceutically acceptable excipient. The pharmaceutically acceptable excipient may be functional molecules as vehicles, adjuvants, carriers, or diluents. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and more preferably, the poly-L-glutamate is present in the pharmaceutical composition containing the CRISPR/Cas-based gene editing system at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. In some embodiments, the DNA vector encoding the CRISPR/Cas-based gene editing system may also include a transfection facilitating agent such as lipids, liposomes, including lecithin liposomes or other liposomes known in the art, as a DNA-liposome mixture (see for example W09324640), calcium ions, viral proteins, polyanions, polycations, or nanoparticles, or other known transfection facilitating agents. Preferably, the transfection facilitating agent is a polyanion, polycation, including poly-L-glutamate (LGS), or lipid.

### 6. METHODS OF DELIVERY

Provided herein is a method for delivering the pharmaceutical formulations of the CRISPR/Cas-based gene editing system for providing genetic constructs and/or proteins of the CRISPR/Cas-based gene editing system. The delivery of the CRISPR/Cas-based gene editing system may be the transfection or electroporation of the CRISPR/Cas-based gene editing system as one or more nucleic acid molecules that is expressed in the cell and delivered to the surface of the cell. The CRISPR/Cas-based gene editing system protein may be delivered to the cell. The nucleic acid molecules may be electroporated using BioRad Gene Pulser Xcell or Amaxa Nucleofector IIb devices or other electroporation device. Several different buffers may be used, including BioRad electroporation solution, Sigma phosphate-buffered saline product # D8537 (PBS). Invitrogen OptiMEM I (OM), or Amaxa Nucleofector solution V (N.V.). Transfections may include a transfection reagent, such as Lipofectamine 2000.

The vector encoding a CRISPR/Cas-based gene editing system protein may be delivered to the mammal by DNA injection (also referred to as DNA vaccination) with and without in vivo electroporation, liposome mediated, nanoparticle facilitated, and/or recombinant vectors. The recombinant vector may be delivered by any viral mode. The viral mode may be recombinant lentivirus, recombinant adenovirus, and/or recombinant adeno-associated virus.

The nucleotide encoding a CRISPR/Cas-based gene editing system protein may be introduced into a cell to induce gene expression of the target gene. For example, one or more nucleotide sequences encoding the CRISPR/Cas-based gene editing system directed towards a target gene may be introduced into a mammalian cell. Upon delivery of the CRISPR/Cas-based gene editing system to the cell, and thereupon the vector into the cells of the mammal, the transfected cells will express the CRISPR/Cas-based gene editing system. The CRISPR/Cas-based gene editing system may be administered to a mammal to induce or modulate gene expression of the target gene in a mammal. The mammal may be human, non-human primate, cow, pig, sheep, goat, antelope, bison, water buffalo, bovids, deer, hedgehogs, anteaters, platypuses, elephants, llama, alpaca, mice, rats, or chicken, and preferably human, cow, pig, or chicken.

Upon delivery of the presently disclosed genetic construct or composition to the tissue, and thereupon the vector into the cells of the mammal, the transfected cells will express the gRNA molecule(s) and the Cas9 molecule. The genetic construct or composition may be administered to a mammal to alter gene expression or to re-engineer or alter the genome. For example, the genetic construct or composition may be administered to a mammal to restore dystrophin function in a mammal. The mammal may be human, non-human primate, cow, pig, sheep, goat, antelope, bison, water buffalo, bovids, deer, hedgehogs, anteaters, platypuses, elephants, llama, alpaca, mice, rats, or chicken, and preferably human, cow, pig, or chicken.

The genetic construct (e.g., a vector) encoding the gRNA molecule(s) and the Cas9 molecule can be delivered to the mammal by DNA injection (also referred to as DNA vaccination) with and without in vivo electroporation, liposome mediated, nanoparticle facilitated, and/or recombinant vectors. The recombinant vector can be delivered by any viral mode. The viral mode can be recombinant lentivirus, recombinant adenovirus, and/or recombinant adeno-associated virus.

A presently disclosed genetic construct (e.g., a vector) or a composition comprising thereof can be introduced into a cell to genetically restore dystrophin function of a dystrophin gene (e.g., human dystrophin gene). In certain embodiments, a presently disclosed genetic construct (e.g., a vector) or a composition comprising thereof is introduced into a myoblast cell from a DMD patient. In certain embodiments, the genetic construct (e.g., a vector) or a composition comprising thereof is introduced into a fibroblast cell from a DMD patient, and the genetically corrected fibroblast cell can be treated with MyoD to induce differentiation into myoblasts, which can be implanted into subjects, such as the damaged muscles of a subject to verify that the corrected dystrophin protein is functional and/or to treat the subject. The modified cells can also be stem cells, such as induced pluripotent stem cells, bone marrow-derived progenitors, skeletal muscle progenitors, human skeletal myoblasts from DMD patients, CD 133⁺ cells, mesoangioblasts, and MyoD- or Pax7-transduced cells, or other myogenic progenitor cells. For example, the CRISPR/Cas-based gene editing system may cause neuronal or myogenic differentiation of an induced pluripotent stem cell.

### 7. ROUTES OF ADMINISTRATION

The CRISPR/Cas-based gene editing system and compositions thereof may be administered to a subject by different routes including, for example, orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian may readily determine the dosing regimen and route of administration that is most appropriate for a particular animal. The CRISPR/Cas-based gene editing system and compositions thereof may be administered by traditional syringes, needleless injection devices, “microprojectile bombardment gone guns,” or other physical methods such as electroporation (“EP”), “hydrodynamic method”, or ultrasound. The composition may be delivered to the mammal by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus.

The presently disclosed genetic constructs (e.g., vectors) or a composition comprising thereof may be administered to a subject by different routes including, for example, orally, parenterally, sublingually, transdermally, rectally, transmucosally, topically via inhalation, via buccal administration, intrapleurally, intravenous, intraarterial, intraperitoneal, subcutaneous, intramuscular, intranasal intrathecal, and intraarticular or combinations thereof. In certain embodiments, the presently disclosed genetic construct (e.g., a vector) or a composition is administered to a subject (e.g., a subject suffering from DMD) intramuscularly, intravenously or a combination thereof. For veterinary use, the presently disclosed genetic constructs (e.g., vectors) or compositions may be administered as a suitably acceptable formulation in accordance with normal veterinary practice. The veterinarian may readily determine the dosing regimen and route of administration that is most appropriate for a particular animal. The compositions may be administered by traditional syringes, needleless injection devices, “microprojectile bombardment gene guns,” or other physical methods such as electroporation (“EP”), “hydrodynamic method”, or ultrasound.

The presently disclosed genetic construct (e.g., a vector) or a composition may be delivered to the mammal by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. The composition may be injected into the skeletal muscle or cardiac muscle. For example, the composition may be injected into the tibialis anterior muscle or tail.

In some embodiments, the presently disclosed genetic construct (e.g., a vector) or a composition thereof is administered by 1) tail vein injections (systemic) into adult mice; 2) intramuscular injections, for example, local injection into a muscle such as the TA or gastrocnemius in adult mice; 3) intraperitoneal injections into P2 mice; or 4) facial vein injection (systemic) into P2 mice.

### 8. CELL TYPES

Any of these delivery methods and/or routes of administration can be utilized with a myriad of cell types, for example, those cell types currently under investigation for cell-based therapies of DMD, including, but not limited to, immortalized myoblast cells, such as wild-type and DMD patient derived lines, primal DMD dermal fibroblasts, induced pluripotent stem cells, bone marrow-derived progenitors, skeletal muscle progenitors, human skeletal myoblasts from DMD patients, CD 133⁺ cells, mesoangioblasts, cardiomyocytes, hepatocytes, chondrocytes, mesenchymal progenitor cells, hematopoetic stem cells, smooth muscle cells, and MyoD- or Pax7-transduced cells, or other myogenic progenitor cells. Immortalization of human myogenic cells can be used for clonal derivation of genetically corrected myogenic cells. Cells can be modified ex vivo to isolate and expand clonal populations of immortalized DMD myoblasts that include a genetically corrected or restored dystrophin gene and are free of other nuclease-introduced mutations in protein coding regions of the genome. Alternatively, transient in vivo delivery of CRISPR/Cas-based systems by non-viral or non-integrating viral gene transfer, or by direct delivery of purified proteins and gRNAs containing cell-penetrating motifs may enable highly specific correction and/or restoration in situ with minimal or no risk of exogenous DNA integration.

### 9. KITS

Provided herein is a kit, which may be used to correct a mutated dystrophin gene and/or restore dystrophin function. The kit comprises at least a gRNA comprising or encoded by a polynucleotide sequence of SEQ ID NO: 19, a complement thereof, a variant thereof, or fragment thereof, or gRNA comprising or encoded by a polynucleotide sequence of SEQ ID NO: 20, a complement thereof, a variant thereof, or fragment thereof, or gRNA targeting a polynucleotide sequence of SEQ ID NO: 17 or SEQ ID NO: 18, a complement thereof, a variant thereof, or fragment thereof, for restoring dystrophin function and instructions for using the CRISPR/Cas-based editing system. Also provided herein is a kit, which may be used for editing of a dystrophin gene in skeletal muscle or cardiac muscle. The kit comprises genetic constructs (e.g., vectors) a composition comprising thereof for genome editing in skeletal muscle or cardiac muscle, as described above, and instructions for using said composition.

Instructions included in kits may be affixed to packaging material or may be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

The genetic constructs (e.g., vectors) or a composition comprising thereof for restoring dystrophin function in skeletal muscle or cardiac muscle may include a modified AAV vector that includes a gRNA molecule(s) and the Cas protein or fusion protein, as described above, that specifically binds and cleaves a region of the dystrophin gene. The CRISPR/Cas-based gene editing system, as described above, may be included in the kit to specifically bind and target a particular region, for example the exon 52, in the mutated dystrophin gene.

### 10. EXAMPLES

The foregoing may be better understood by reference to the following examples, which are presented for purposes of illustration and are not intended to limit the scope of the invention. The present disclosure has multiple aspects and embodiments, illustrated by the appended non-limiting examples.

### Example 1

### SaCas9 gRNA Design and Screening for hDMD-Intron51 Target in HEK293T Cells

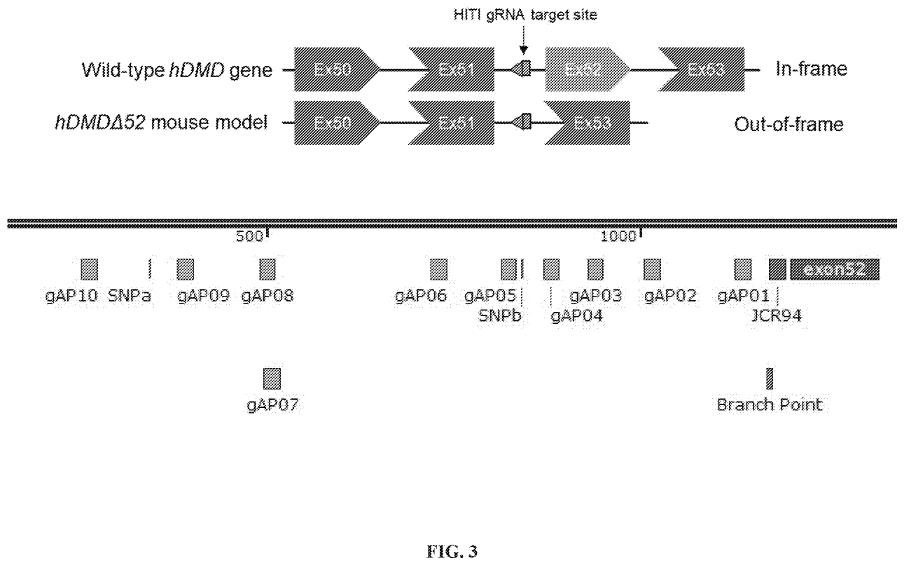

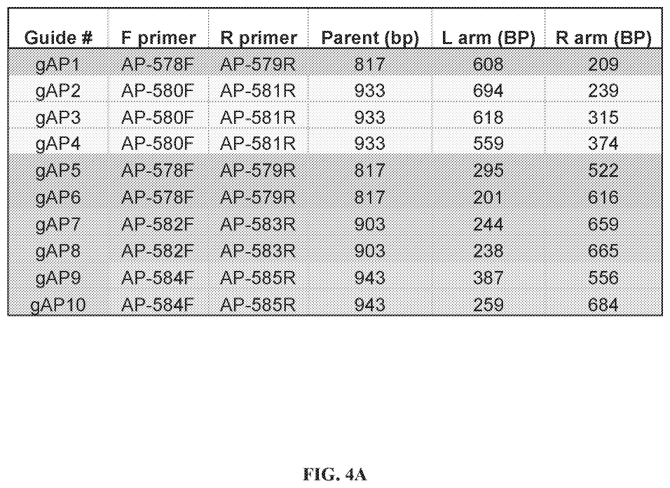

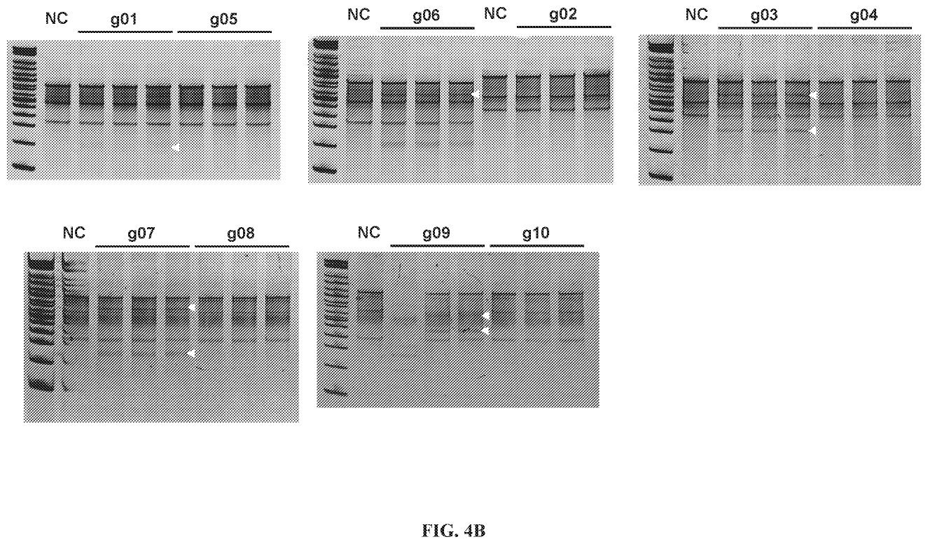

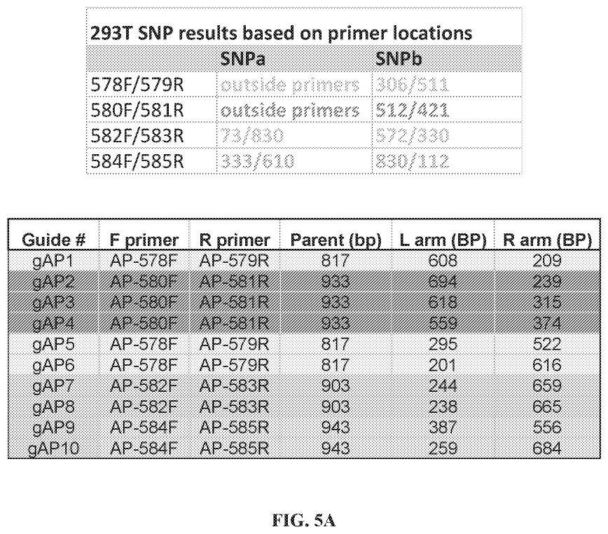

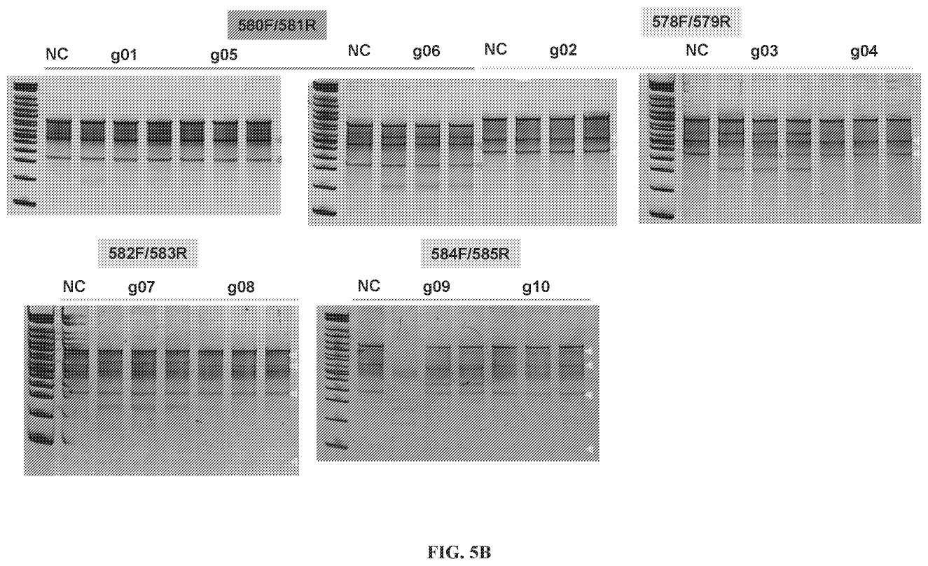

SaCas9 gRNAs targeting hDMD-Intron51, upstream of hDMD-Exon52, were designed with 21 bp spacers and cloned into individual expression plasmids (FIG. 3). HEK293T cells in a 24-well plate were transfected with a plasmid expressing SaCas9 under a CMV promoter (375 ng) and a plasmid expressing individual gRNAs under a U6 promoter (125 ng). Genomic DNA was extracted 3 days post transfection. Editing efficiency was evaluated by surveyor analysis (FIG. 4A and FIG. 4B). Negative control (NC) contained no gRNA. Extra bands relating to single-nucleotide polymorphisms (SNPs) in HEK293T genomic DNA were also observed. These bands corresponded to the expected sizes based on SNP locations (FIG. 5A, FIG 5B). Testing was continued for 19-23 bp spacers for gRNA 03, gRNA 06, gRNA 07, and gRNA 09.

### Example 2

### SaCas9 gRNA Design and Screening for hDMD-Intron51 Target in Human 8036 Myoblasts

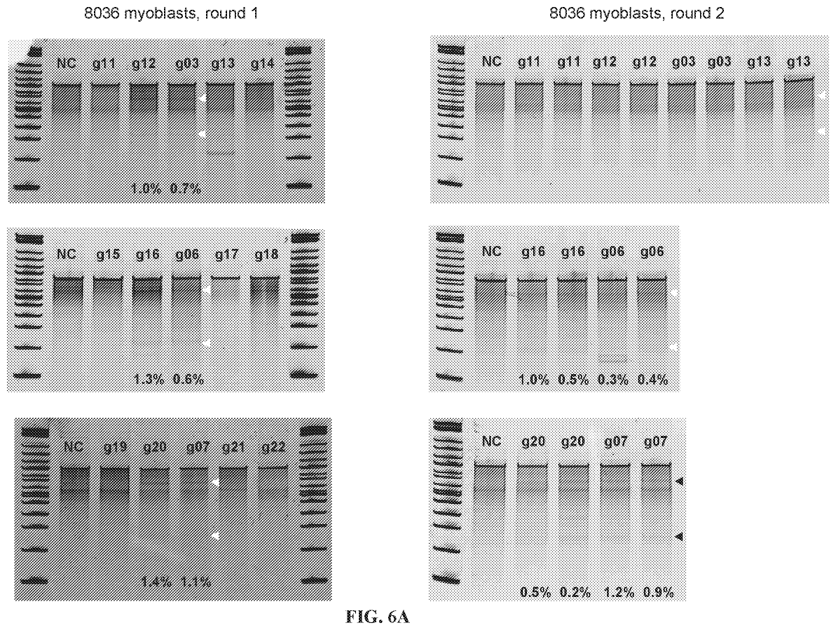

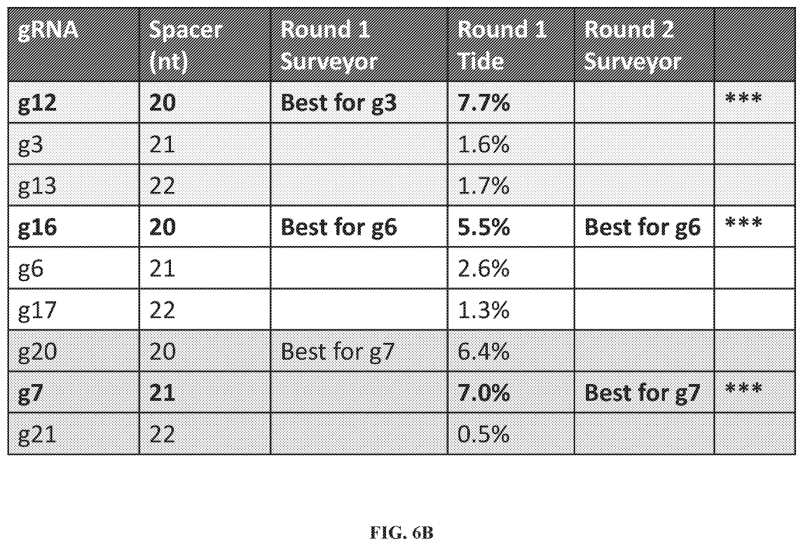

Based on the editing activity of gRNAs tested in HEK293Ts, the top gRNAs were redesigned with 19-23 bp spacers and cloned into individual expression plasmids. The redesigned gRNAs were screened in Human 8036 myoblasts. Human 8036 myoblasts in 6-well plates were electroporated with a plasmid expressing SaCas9 under a CMV promoter (10 μg) and a plasmid expressing individual gRNAs under a U6 promoter (10 μg). Genomic DNA was isolated at 3 days post electroporation. Editing efficiency was evaluated by surveyor analysis (FIG. 6A, FIG. 6B). Negative control (NC) contained no gRNA. Editing efficiency was also evaluated by tide analysis. gRNAs g12, g16, and g7 were selected to generate AAV-integration vectors.

### Example 3

### SaCas9 gRNA Screening for AAV-HITI Donor Plasmids in HEK293T Cells

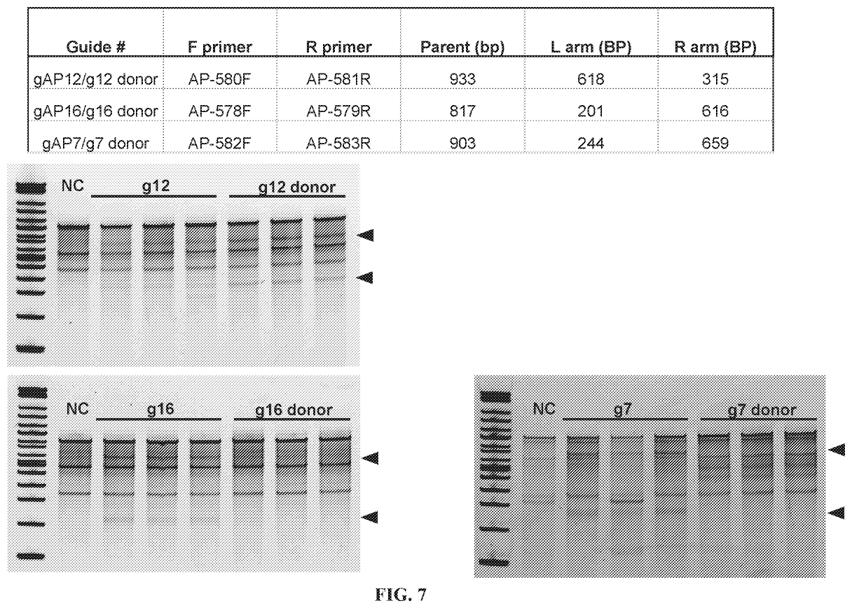

Based on the editing activity of gRNAs tested in human 8036 myoblasts, the top gRNAs were cloned into AAV-HITI donor plasmids (gRNAs g12, g16, and g7). HEK293T cells in a 24-well plate were transfected with a plasmid expressing SaCas9 under a CMV promoter (375 ng) and a plasmid expressing individual gRNAs under a U6 promoter (125 ng) or an AAV-HITI donor plasmid expressing individual gRNAs under a U6 promoter (125 ng). Genomic DNA was extracted 3 days post transfection. Editing efficiency was evaluated by surveyor analysis (FIG. 7). Based on the editing activity of the AAV-HITI donor plasmids expressing individual gRNAs, the g7 and g12 donors were used in future experiments, to generate AAV-HITI donor plasmids.

### Example 4

### In Vitro HITI-Mediated Integration of hDMD-Exon52

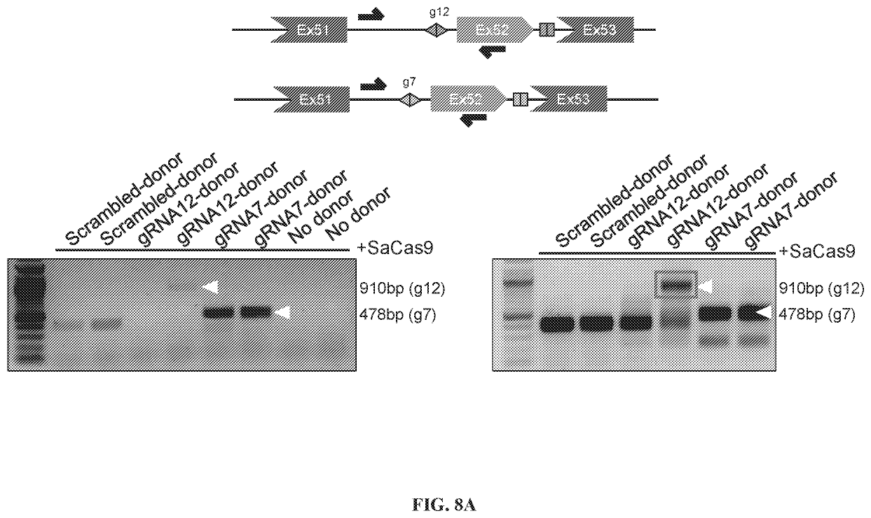

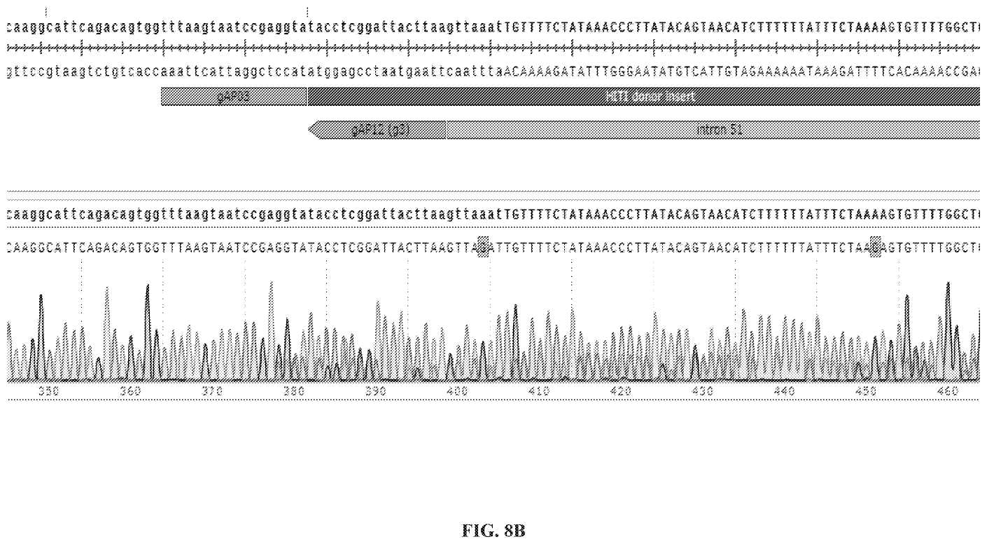

Primary myoblasts were isolated from hDMDΔ52/mdx mice. These cells in a 6-well plate were electroporated with an AAV-CMV-Cas9 plasmid (10 μg) and an AAV-U6-gRNA-Ex52 donor plasmid expressing individual gRNAs (10 μg). Genomic DNA was extracted 6 days post electroporation. Nested PCR was used to detect HITI-mediated hDMD-Exon52 integration (FIG. 8A). The boxed band in the gRNA12-donor sample was excised and sent for Sanger sequencing (FIG. 8B) to confirm integration of the hDMD-Exon52 donor at the targeted site.

### Example 5

### In Vivo HITI-Mediated Integration of hDMD-Exon52 in hDMDΔ52/mdx Mouse Model

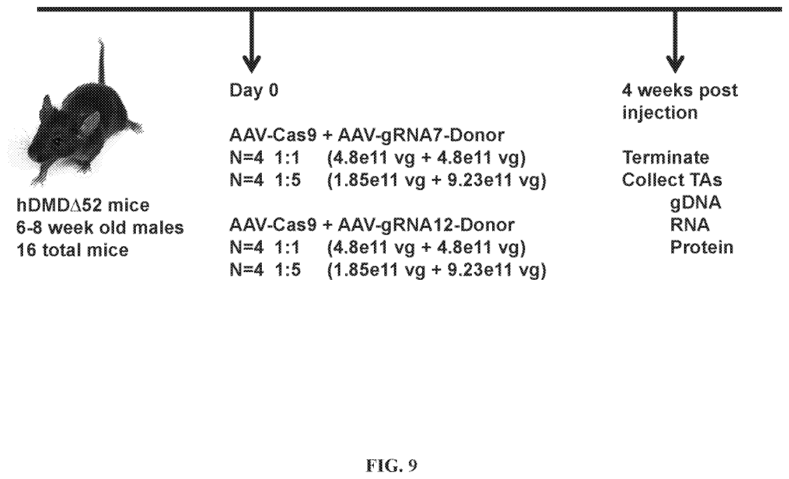

Shown in FIG. 9 is a schematic diagram of the experiments used to confirm in vivo editing, determine the best gRNA/donor sequence combination, and determine the best ratio of AAV-Cas9 to AAV-donor plasmids. Male 6-8 week old hDMDΔ52/mdx mice were injected with AAV-Cas9 and AAV-HITI donors via local intramuscular injection in the tibialis anterior (TA) muscle. At 4 weeks post injection, the TA muscle was collected for processing to evaluate HITI-mediated editing. PBS injected mice served as negative controls; N=4.

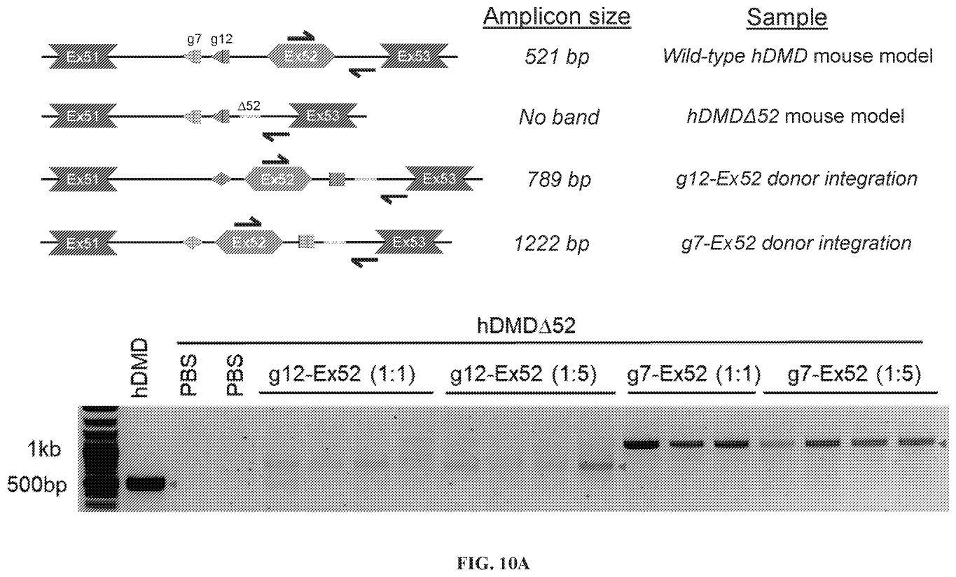

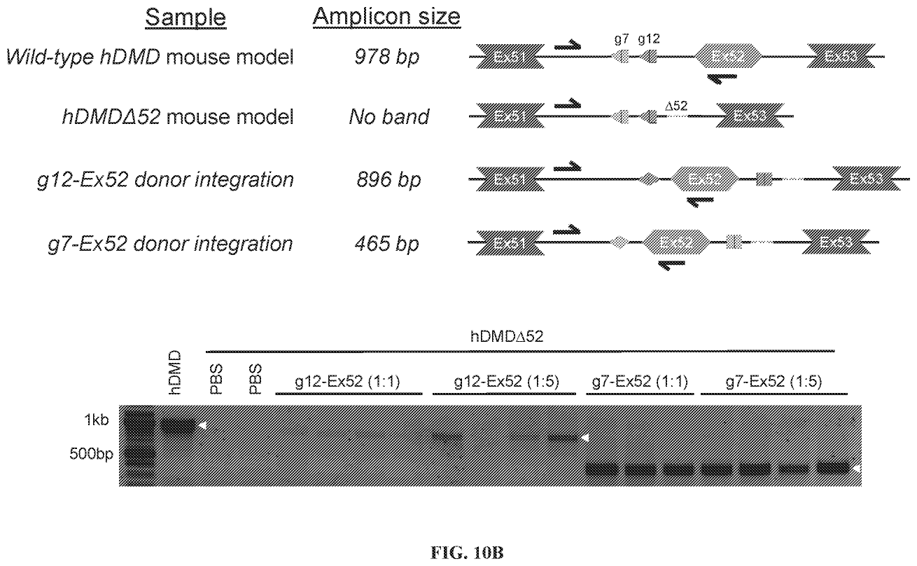

Targeted Ex52 insertion in the genomic DNA of hDMDΔ52/mdx mice was examined with primers downstream of the targeted cut site (FIG. 10A) and with primers upstream of the targeted cut site (FIG. 10B). Genomic DNA was extracted from TA muscle. PCR analysis confirmed the presence of Ex52 insertion at the targeted site.

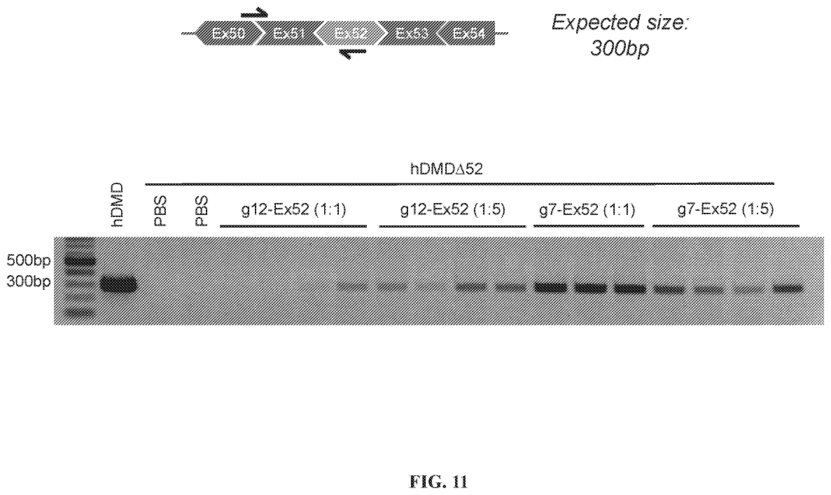

Targeted Ex52 insertion in mRNA of treated hDMDΔ52/mdx mice was examined (FIG. 11). Total RNA was extracted from TA muscle and used to generate cDNA. PCR analysis confirmed the presence of Ex52 insertion via splicing in RNA transcripts.

### Example 6

### Dystrophin Protein Restoration of Treated hDMDΔ52/mdx Mice

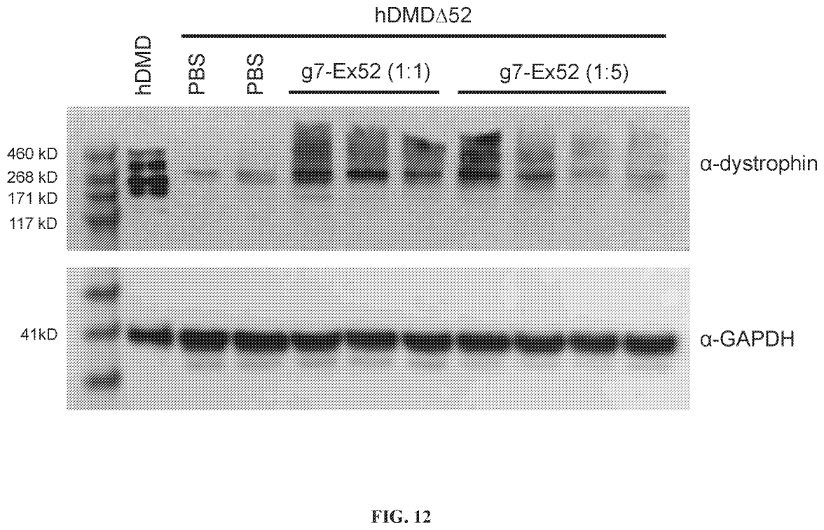

Protein was extracted from TA muscle and used for Western blot analysis. For PBS and treated TA muscles, 25 μg total protein was loaded. To serve as a positive control, 3.125 μg of total protein from hDMD/mdx TA muscle was loaded. The membrane was stained with anti-dystrophin (clone 2c6; MANDYS106) or anti-GAPDH (clone 14C10). Western blot analysis confirmed protein restoration in treated mice (FIG. 12).

### Example 7

### Deep Sequencing Quantification of AAV-ITR Sequence Integration in Edited hDMDΔ52/mdx Mice

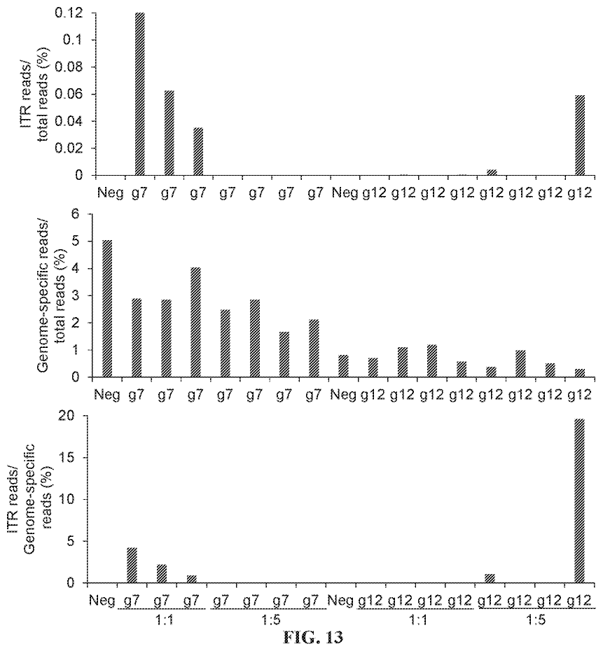

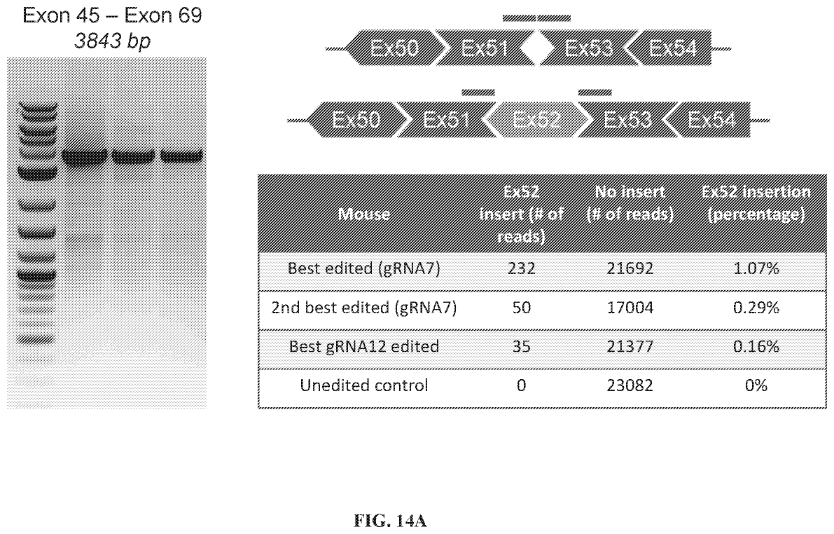

Shown in FIG. 13 are results from Illumina deep sequencing quantification of exon 52 genomic integration in edited mice. Genomic DNA was extracted from TA muscle. For unbiased sequencing analysis, genomic DNA was tagmented using a Nextera Tn5 transposon. To enrich the targeted sequence, PCR was completed using a genome specific primer upstream of the Intron51 target site and was paired with a reverse primer specific for the tag sequence inserted by the transposon. A second PCR was used to add experimental barcodes and Illumina adapter sequences. ITR integration was detected by next-generation sequencing. Bowtie analysis was used to detect the presence of ITR sequences matching the AAV vectors, and genome-specific sequences that matched the genomic DNA sequence between the genome-specific primer and intron 51 target site.

### Example 8

### PacBio Sequencing Quantification of Exon52 Insertion in mRNA of Edited hDMDΔ52/mdx Mice

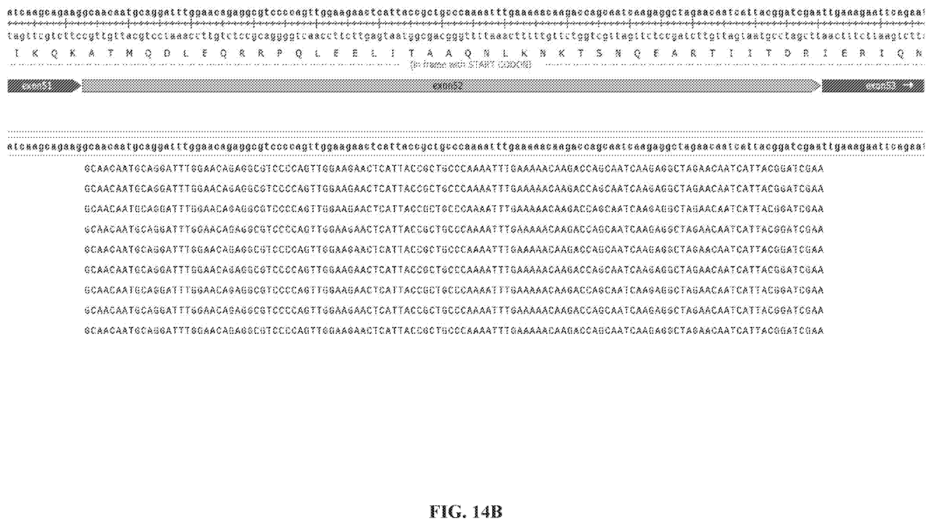

Total RNA was extracted from TA muscle and used to generate cDNA (FIG. 14A). To enrich the targeted sequence, PCR was completed using primers in Exon45 and Exon69. A second PCR was used to add experimental barcodes and PacBio adapter sequences. Exon52 insertion was detected by PacBio sequencing (FIG. 14B). Reads with 118 nt between 3′-Exon51 and 5′-Exon53 sequences were quantified. These sequences were aligned to the Exon52 donor for confirmation of the intended edit. Sequencing reads with 118 bp between Exon51 and Exon53 matched the Exon52 sequence.

For reasons of completeness, various aspects are set out in the following numbered clause:

Clause 1. A CRISPR/Cas-based genome editing system comprising one or more vectors encoding a composition, the composition comprising: (a) a guide RNA (gRNA) targeting a fragment of a mutant dystrophin gene; (b) a Cas protein or a fusion protein comprises the Cas protein; and (c) a donor sequence comprising a fragment of a wild-type dystrophin gene.

Clause 2. A CRISPR/Cas-based genome editing system comprising: (a) a guide RNA (gRNA) targeting a fragment of a mutant dystrophin gene; (b) a Cas protein or a fusion protein comprises the Cas protein; and (c) a donor sequence comprising a fragment of a wild-type dystrophin gene.

Clause 3. The system of clause 1 or 2, wherein the fragment of the wild-type dystrophin gene is flanked by two gRNA spacers and/or PAM sequences.

Clause 4. The system of any one of clauses 1-3, wherein the gRNA targets an intron that is juxtaposed with an exon of the mutant dystrophin gene, and wherein the exon is selected from exons 1-8, 10, 11, 12, 14, 16-22, 43-59, and 61-66 of the mutant dystrophin gene.

Clause 5. The system of any one of clauses 1-3, wherein the donor sequence comprises an exon of the wild-type dystrophin gene or a functional equivalent thereof, and wherein the exon is selected from exons 1-8, 10, 11, 12, 14, 16-22, 43-59, and 61-66 of the wild-type dystrophin gene.

Clause 6. The system of clause 4, wherein the exon of the mutant dystrophin gene is mutated or at least partially deleted from the dystrophin gene, or wherein the exon of the mutant dystrophin gene is deleted and the intron is juxtaposed to where the deleted exon would be in a corresponding wild-type dystrophin gene.

Clause 7. The system of clause 4 or 5, wherein the exon is exon 52.

Clause 8. The system of any one of clauses 1-7, wherein the gRNA binds and targets a polynucleotide sequence comprising: a) SEQ ID NO: 17 or SEQ ID NO: 18; b) a fragment of SEQ ID NO: 17 or SEQ ID NO: 18; c) a complement of SEQ ID NO: 17 or SEQ ID NO: 18, or fragment thereof; d) a nucleic acid that is substantially identical to SEQ ID NO: 17 or SEQ ID NO: 18, or complement thereof; or e) a nucleic acid that hybridizes under stringent conditions to SEQ ID NO: 17 or SEQ ID NO: 18, complement thereof, or a sequence substantially identical thereto.

Clause 9. The system of any one of clauses 1-8, wherein the gRNA comprises or is encoded by a polynucleotide sequence of SEQ ID NO: 19 or SEQ ID NO: 20, or variant thereof.

Clause 10. The system of any one of clauses 1-9, wherein the Cas protein is a Streptococcus pyogenes Cas9 protein or a Staphylococcus aureus Cas9 protein.

Clause 11. The system of any one of clauses 1-10, wherein the Cas protein comprises an amino acid sequence of SEQ ID NO: 1, 2, 3, or 4.

Clause 12. The system of any one of clauses 3-11, wherein the two gRNA spacers independently comprise a sequence selected from SEQ ID NO: 5-8 and 25-45.

Clause 13. The system of clause 12, wherein the two gRNA spacers are identical.

Clause 14. The system of clause 12, wherein the two gRNA spacers are different.

Clause 15. The system of any one of clauses 3-14, wherein at least one of the two gRNA spacers comprises a sequence of SEQ ID NO: 25 or SEQ ID NO: 26.

Clause 16. The system of any one of clauses 1-15, wherein the donor sequence comprises the polynucleotide of SEQ ID NO: 21 or SEQ ID NO: 22.

Clause 17. The system of any one of clauses 1 and 3-16, wherein the vector is a viral vector.

Clause 18. The system of clause 17, wherein the vector is an Adeno-associated virus (AAV) vector.

Clause 19. The system of clause 18, wherein the AAV vector is AAV1, AAV2, AAV3, AAV4, AAV5, AAV6, AAV7, AAV8, AAV9, AAV-10, AAV-11, AAV-12, AAV-13, or AAVrh.74 vector.

Clause 20. The system of clause 18, wherein one of the one or more vectors comprises the polynucleotide sequence of SEQ ID NO: 23 or 24.

Clause 21. The system of any one of clauses 1-20, wherein the molar ratio between gRNA and donor sequence is 1:1, or 1:15, or from 5:1 to 1:10, or from 1:1 to 1:5.

Clause 22. A recombinant polynucleotide encoding a donor sequence comprising a fragment of a wild-type dystrophin gene or a functional equivalent thereof, and wherein the fragment or functional equivalent thereof is flanked by two gRNA spacers.

Clause 23. The recombinant polynucleotide of clause 22, wherein the donor sequence comprises an exon of the dystrophin gene, and wherein the exon is selected from exons 1-8, 10, 11, 12, 14, 16-22, 43-59, and 61-66.

Clause 24. The recombinant polynucleotide of clause 22 or 23, wherein the recombinant polynucleotide comprises a sequence of SEQ ID NO: 23 or 24.

Clause 25. A vector comprising the recombinant polynucleotide of any one of clauses 22-24.

Clause 26. The vector of clause 25, wherein the vector comprises a heterologous promoter driving expression of the recombinant polynucleotide.

Clause 27. A cell comprising the recombinant polynucleotide of any one of clauses 22-24 or the vector of clause 25 or 26.

Clause 28. A composition for restoring dystrophin function in a cell having a mutant dystrophin gene, the composition comprising the system of any one of clauses 1-21, the recombinant polynucleotide of any one of clauses 22-24, or the vector of clause 25 or 26.

Clause 29. A kit comprising the system of any one of clauses 1-21, the recombinant polynucleotide of any one of clauses 22-24, or the vector of clause 25 or 26, or the composition of clause 28.

Clause 30. A method for restoring dystrophin function in a cell or a subject having a mutant dystrophin gene, the method comprising contacting the cell or the subject with the system of any one of clauses 1-21, the recombinant polynucleotide of any one of clauses 22-24, or the vector of clause 25 or 26, or the composition of clause 28.

Clause 31. The method of clause 30, wherein the dystrophin function is restored by insertion of exon 52 of the wild-type dystrophin gene.

Clause 32. The method of clause 30 or 31, wherein the subject is suffering from Duchenne Muscular Dystrophy.

Clause 33. A method for restoring dystrophin function in a cell or a subject having a disrupted dystrophin gene caused by one or more deleted or mutated exons, the method comprising contacting the cell or the subject with the system of any one of clauses 1-21, the recombinant polynucleotide of any one of clauses 22-24, or the vector of clause 25 or 26, or the composition of clause 28.

Clause 34. The method of clause 33, wherein dystrophin function is restored by inserting one or more wild-type exons of dystrophin gene corresponding to the one or more deleted or mutated exons.

Clause 35. The method of clause 34, wherein one of the deleted or mutated exons is exon 52.

SEQUENCES

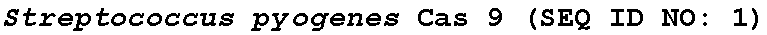

MDKKYSIGLDIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLFKTNRK
VTVKQLKEDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPAIKKGILQTVKVV
DELVKVMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGITIMERSSFEKNP
IDFLEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSITGLYETRIDLSQ
LGGD

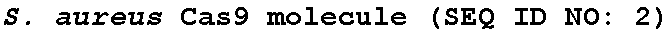

MKRNYILGLDIGITSVGYGIIDYETRDVIDAGVRLFKEANVENNEGRRSKRGARRLKRRRH
RIQRVKKLLFDYNLLTDHSELSGINPYEARVKGLSQKLSEEEFSAALLHLAKRRGVHNVNEV
EEDTGNELSTKEQISRNSKALEEKYVAELQLERLKKDGEVRGSINRFKTSDYVKEAKQLLKV
QKAYHQLDQSFIDTYIDLLETRRTYYEGPGEGSPFGWKDIKEWYEMLMGHCTYFPEELRSVK
YAYNADLYNALNDLNNLVITRDENEKLEYYEKFQIIENVFKQKKKPTLKQIAKEILVNEEDI
KGYRVTSTGKPEFTNLKVYHDIKDITARKEIIENAELLDQIAKILTIYQSSEDIQEELTNLN
SELTQEEIEQISNLKGYTGTHNLSLKAINLILDELWHTNDNQIAIFNRLKLVPKKVDLSQQK
EIPTTLVDDFILSPVVKRSFIQSIKVINAIIKKYGLPNDIIIELAREKNSKDAQKMINEMQK
RNRQTNERIEEIIRTTGKENAKYLIEKIKLHDMQEGKCLYSLEAIPLEDLLNNPFNYEVDHI
IPRSVSFDNSFNNKVLVKQEENSKKGNRTPFQYLSSSDSKISYETFKKHILNLAKGKGRISK
TKKEYLLEERDINRFSVQKDFINRNLVDTRYATRGLMNLLRSYFRVNNLDVKVKSINGGFTS
FLRRKWKFKKERNKGYKHHAEDALIIANADFIFKEWKKLDKAKKVMENQMFEEKQAESMPEI
ETEQEYKEIFITPHQIKHIKDFKDYKYSHRVDKKPNRELINDTLYSTRKDDKGNTLIVNNLN
GLYDKDNDKLKKLINKSPEKLLMYHHDPQTYQKLKLIMEQYGDEKNPLYKYYEETGNYLTKY
SKKDNGPVIKKIKYYGNKLNAHLDITDDYPNSRNKVVKLSLKPYRFDVYLDNGVYKFVTVKN
LDVIKKENYYEVNSKCYEEAKKLKKISNQAEFIASFYNNDLIKINGELYRVIGVNNDLLNRI
EVNMIDITYREYLENMNDKRPPRIIKTIASKTQSIKKYSTDILGNLYEVKSKKHPQIIKKG

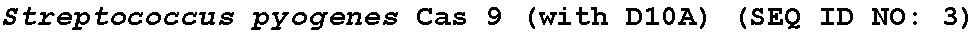

MDKKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLFKTNRK
VTVKQLKEDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPAIKKGILQTVKVV
DELVKVMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFKVREINNYHHAHDAYLNAV
VGTALIKKYPKLESEFVYGDYKVYDVRKMIAKSEQEIGKATAKYFFYSNIMFFKTEITLAN
GEIRKRPLIETNGETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGITIMERSSFEKNP
IDFLEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSITGLYETRIDLSQ
LGGD

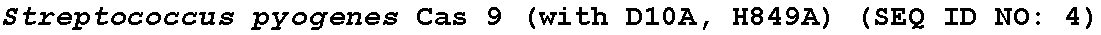

MDKKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLFKTNRK
VTVKQLKEDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPAIKKGILQTVKVV
DELVKVMGRHKPENIVIEMARENQTTQKGQKNSRERMKRIEEGIKELGSQILKEHPVENTQL
QNEKLYLYYLQNGRDMYVDQELDINRLSDYDVDAIVPQSFLKDDSIDNKVLTRSDKNRGKSD
NVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGITIMERSSFEKNP
IDFLEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSITGLYETRIDLSQ
LGGD
PAM (SEQ ID NO: 9)
ATTCCT
PAM (SEQ ID NO: 10)
NGG
PAM (SEQ ID NO: 11)
NNNRRT
PAM (SEQ ID NO: 12)
NNGRR (R=A or G)
PAM (SEQ ID NO: 13)
NNGRRN (R=A or G)
PAM (SEQ ID NO: 14)
NNGRRT (R=A or G)
PAM (SEQ ID NO: 15)
NNGRRV (R=A or G; V=A,C,or G)
PAM (SEQ ID NO: 16)
NGA
Target for gRNA7 (SEQ ID NO: 17)
TCATTTATAATACAGGGGAAT
Target for gRNA12 (SEQ ID NO: 18)
TTAAGTAATCCGAGGTACTC
gRNA7 (includes target sequence and scaffold) (SEQ ID NO: 19)
TCATTTATAATACAGGGGAATGTTTTAGTACTCTGGAAACAGAATCTACTAAAACAAGGCAA
AATGCCGTGTTTATCTCGTCAACTTGTTGGCGAGA
gRNA12 (includes target sequence and scaffold) (SEQ ID NO: 20)
TTAAGTATCCGAGGTACTCGTTTTAGTACTCTGGAAACAGAATCTACTAAAACAAGGCAAA
ATGCCGTGTTTATCTCGTCAACTTGTTGGCGAGA
Exon 52 (SEQ ID NO: 21)
GCAACAATGCAGGATTTGGAACAGAGGCGTCCCCAGTTGGAAGAACTCATTACCGCTGCCCA
AAATTTGAAAAACAAGACCAGCAATCAAGAGGCTAGAACAATCATTACGGATCGAA
Exon 52 with some intron (SEQ ID NO: 22)
GTTAAATTGTTTTCTATAAACCCTTATACAGTAACATCTTTTTTATTTTCTAAAAGTGTTTTG
GCTGGTCTCACAATTGTACTTTACTTTGTATTATGTAAAAGGAATACACAACGCTGAAGAAC
CCTGATACTAAGGGATATTTGTTCTTACAGGCAACAATGCAGGATTTGGAACAGAGGCGTCC
CCAGTTGGAAGAACTCATTACCGCTGCCCAAAATTTGAAAAACAAGACCAGCAATCAAGAGG
CTAGAACAATCATTACGGATCGAAGTAAGTTTTTTAACAAGCATGGGACACACAAAGCAAGA
TGCATGACAAGTTTCAATAAAAACTTAAGTTCATATATCCCCCTCACATTTATAAAAATAAT
GTGAAATAATTGTAAATGATAACAATTGTGCTGAGATTTTCAGTCCATAATGTTACCTTTTA
ATAAATGAATGTAATTCCATTGAATAGAAGAAATAC
AAV for gRNA7

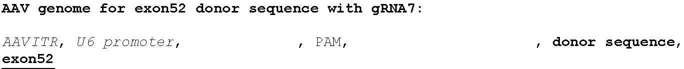

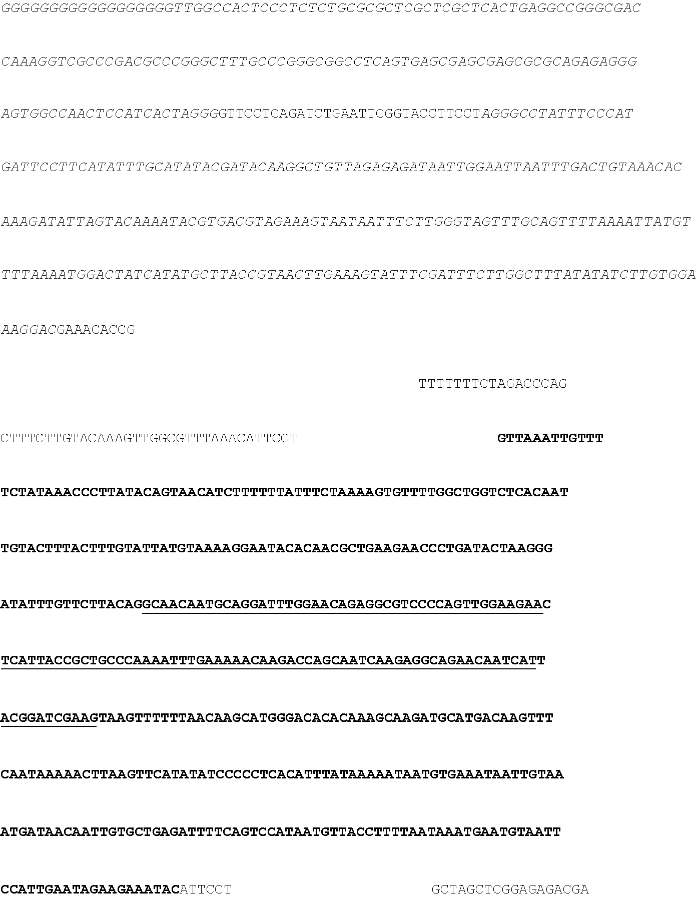

CATCTAGAATTAAACTGTCACTATCGATTACTAATTTTTTGCTCATAATAGAAGCAGCGATTAAAG
GAATAGAATCAACAGTTCCAGTAACATCTCTTAGTGCATACATTTTTTTATCAGCAGGAACAATAT
CATCTGACTGACCTGTGATGCTCATTCCAACTTCATTAATTGTTTTAATGAATTTTTCTTTAGATAAT
TCACTTGTTCAACCTTTACATGACTCTAATTTATCAATAGTACCACCAGTTACTCAAGTCCTCTAC
CAGAAAGTTTACAAACCTTTACTCCATAACTTGCAACTAACGGACTATATACTAAACTTGTTTTGTC
TCCAACTCCGCCAGTTGAATGCTTATCAGCTTTTAAACCTGTAACCTCACTGACATCATAAACATAT
CCTGATTCAACATAAGATTGGGTTAATGCTAAAGTTTCTGCTTTGGTCATTCCATTAAAATAAACC
GCCATAGCAAAAGCAGCCATTTGATAATCTGTTACATTATTTTTAACGTAACTGTTTATCAATCATT
TAATTTCTTCAGCTGATAATTCTATTGAATGTTTCTTTTTTTCTATAATTTCACTAAAACTGTAGTTC
ATAAGTCTCCTTTTGTAAGAGTGCACAATATTTACACCATTACTCTTTCTACTATATTATAATAGAA
TAGACATATAAAAAACATAAGGAGTACAAATGGTTTTTGATAAAAATAACAAAGTTTATAGTGAA
TGAATAAATAGCCAAAAATTGGATGATGAGTTGAAAAGCCTTTTAGTAAATGCTACTGATGATGA
ATTGCATGCAGCATTTGAAGGAATAGAGTTAGAATTTGGAACAGCAGGTATAAGAGGTATTCTTG
GAGCAGGACCTGGAAGATTTAACGTTTACACTGTTAAAAAAGTTACTATTGCATTTGCAGAATTAT
TAAAACAAAATTACCCAAATAGGTTGAATGATGGAATAGTTGTTGGTCATGATAACCGTCATAATT
CTAAACAGTTTGCAAAAGTTGTAGCCGAAGTTTTATCAAGCTTGTGAAATAGCTGTTGAAGCTGGA
TTAGAATTTGTTAAAACATCAACAGGATTTTCAAAATCAGGTGCAACATTTGAAGATGTTAAACTA
ATGAAGTCAGTTGTTAAAGACAATGCTTTAGTTAAAGCAGCTGGTGGAGTTAGAACATTTGAAGAT
GCTCAAAAAATGATTGAAGCAGGAGCTGACCGCTTAGGAACAAGTGGTGGAGTAGCTATTATTAA
AGGTGAAGAAAACAACGCGAGTTACTAAAACTAGCGTTTTTTTATTTTGCTCATTTTTATTAAAAG
TTTGCAAAAAGGAACATAAAAATTCTAATTATTGATACTAAAGTTATTAAAAAGAAGATTTTGGTT
GATTTTATAAAGGTCATAGAATATAATATTTTAGCATGTGTATTTTGTGTGCTCATTTACAACCGTG
TCGCGGCCGCGGGGATCCAGACATGATAAGATACATTGATGAGTTTGGACAAACCACAACTAGAA
TGCAGTGAAAAAAATGCTTTATTTGTGAAATTTGTGATGCTATTGCTTTATTTGTAACCATTATAAG
CTGCAATAAACAAGTTAACAACAACAATTGCATTCATTTTATGTTTCAGGTTCAGGGGGAGGTGTG
GGAGGTTTTTTAGTCGACCTCGAGCAGTGTGGTTTTGCAAGAGGAAGCAAAAAGCCTCTCCACCCA
GGCCTGGAATGTTTCCACCCAAGTCGAAGGCAGTGTGGTTTTGCAAGAGGAAGCAAAAAGCCTCT
CCACCCAGGCCTGGAATGTTTCCACCCAATGTCGAGCAACCCCGCCCAGCGTCTTGTCATTGGCGA
ATTCGAACACGCAGATGCAGTCGGGGCGGCGCGGTCCCAGGTCCACTTCGCATATTAAGGTGACG
CGTGTGGCCTCGAACACCGAGCGACCCTGCAGCCAATATGGGATCGGCCATTGAACAAGATGGAT
TGCACGCAGGTTCTCCGGCCGCTTGGGTGGAGAGGCTATTCGGCTATGACTGGGCACAACAGACA
ATCGGCTGCTCTGATGCCGCCGTGTTCCGGCTGTCAGCGCAGGGGCGCCCGGTTCTTTTTGTCAAG
ACCGACCTGTCCGGTGCCCTGAATGAACTGCAGGACGAGGCAGCGCGGCTATCGTGGCTGGCCAC
GACGGGCGTTCCTTGCGCAGCTGTGCTCGACGTTGTCACTGAAGCGGGAAGGGACTGGCTGCTATT
GGGCGAAGTGCCGGGGCAGGATCTCCTGTCATCTCACCTTGCTCCTGCCGAGAAAGTATCCATCAT
GGCTGATGCAATGCGGCGGCTGCATACGCTTGATCCGGCTACCTGCCCATTCGACCACCAAGCGAA
ACATCGCATCGAGCAGCACGTACTCGGATGGAAGCCGGTCTTGTCGATCAGGATGATCTGGACG
AAGAGCATCAGGGGCTCGCGCCAGCCGAACTGTTCGCCAGGCTCAAGGCGCGCATGCCCGACGGC
GAGGATCTCGTCGTGACCCATGGCGATGCCTGCTTGCCGAATATCATGGTGGAAAATGGCCGCTTT
TCTGGATTCATCGACTGTGGCCGGCTGGGTGTGGCGGACCGCTATCAGGACATAGCGTTGGCTACC
CGTGATATTGCTGAAGAGCTTGGCGGCGAATGGGCTGACCGCTTCCTCGTGCTTTACGGTATCGCC
GCTCCCGATTCGCAGCGCATCGCCTTCTATCGCCTTCTTGACGAGTTCTTCTGAGGGGATCCGTCGA
CTAGAGCTCGCTGATCAGCCTCGACTGTGCCTTCTAGTTGCCAGCCATCTGTTGTTTGCCCCTCCCC
CGTGCCTTCCTTGACCCTGGAAGGTGCCACTCCCACTGTCCTTTCCTAATAAAATGAGGAAATTGC
ATCGCATTGTCTGAGTAGGTGTCATTCTATTCTGGGGGGTGGGGTGGGGCAGGACAGCAAGGGGG
AGGATTGGGAAGACAATAGCAGGCATGCTGGGGAGAGATCTAGGAACCCCTAGTGATGGAGTTGG
CCACTCCCTCTCTGCGCGCTCGCTCGCTCACTGAGGCCGCCCGGGCAAAGCCGGGCGTCGGGCGACC
TTTGGTCGCCCGGCCTCAGTGAGCGAGCGAGCGCGCAGAGAGGGAGTGGCCAACCCCCCCCCCCCCC
CCCC
AAV for gRNA12 (SEQ ID NO: 24)

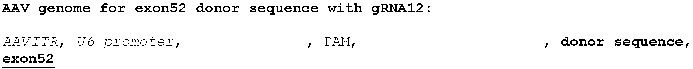

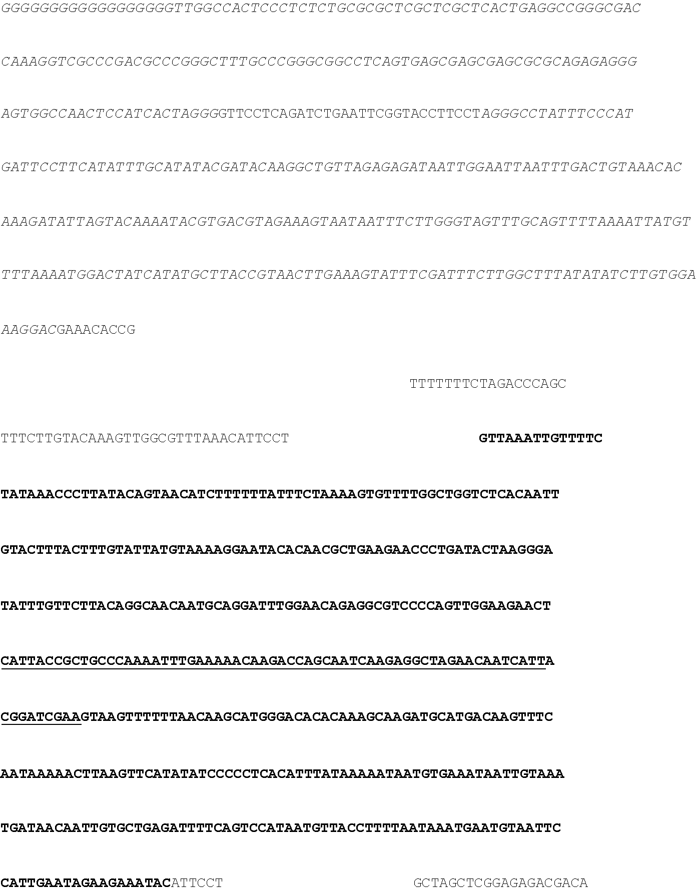

TCTAGAATTAAACTGTCACTATCGATTACTAATTTTTTGCTCATAATAGAAGCAGCGATTAAAGGA
ATAGAATCAACAGTTCCAGTAACATCTCTTAGTGCATACATTTTTTTATCAGCAGGAACAATATCA
TCTGACTGACCTGTGATGCTCATTCCAACTTCATTAATTGTTTTAATGAATTTTTCTTTAGATAATTC
ACTTGTTCAACCTTTACATGACTCTAATTTATCAATAGTACCACCAGTTACTCCAAGTCCTCTACCA
GAAAGTTTACAAACCTTTACTCCATAACTTGCAACTAACGGACTATATACTAAACTTGTTTTGTCTC
CAACTCCGCCAGTTGAATGCTTATCAGCTTTTAAACCTGTAACCTCACTGACATCATAAACATATC
CTGATTCAACATAAGATTGGGTTAATGCTAAAGTTTCTGCTTTGGTCATTCCATTAAAATAAACCG
CCATAGCAAAAGCAGCCATTTGATAATCTGTTACATTATTTTTAACGTAACTGTTTATCAATCATTT
AATTTCTTCAGCTGATAATTCTATTGAATGTTTCTTTTTTTCTATAATTTCACTAAAACTGTAGTTCA
TAAGTCTCCTTTTGTAAGAGTGCACAATATTTACACCATTACTCTTTCTACTATATTATAATAGAAT
AGACATATAAAAAACATAAGGAGTACAAATGGTTTTTGATAAAAATAACAAAGTTTATAGTGAAT
GAATAAATAGCCAAAAATTGGATGATGAGTTGAAAAGCCTTTTAGTAAATGCTACTGATGATGAA
TTGCATGCAGCATTTGAAGGAATAGAGTTAGAATTTGGAACAGCAGGTATAAGAGGTATTCTTGG
AGCAGGACCTGGAAGATTTAACGTTTACACTGTTAAAAAAGTTACTATTGCATTTGCAGAATTATT
AAAACAAAATTACCCAAATAGGTTGAATGATGGAATAGTTGTTGGTCATGATAACCGTCATAATTC
TAAACAGTTTGCAAAAGTTGTAGCCGAAGTTTTATCAAGCTTGTGAAATAGCTGTTGAAGCTGGAT
TAGAATTTGTTAAAACATCAACAGGATTTTCAAAATCAGGTGCAACATTTGAAGATGTTAAACTAA
TGAAGTCAGTTGTTAAAGACAATGCTTTAGTTAAAGCAGCTGGTGGAGTTAGAACATTTGAAGATG
CTCAAAAAATGATTGAAGCAGGAGCTGACCGCTTAGGAACAAGTGGTGGAGTAGCTATTATTAAA
GGTGAAGAAAACAACGCGAGTTACTAAAACTAGCGTTTTTTTATTTTGCTCATTTTTATTAAAAGTT
TGCAAAAAGGAACATAAAAATTCTAATTATTGATACTAAAGTTATTAAAAAGAAGATTTTGGTTGA
TTTTATAAAGGTCATAGAATATAATATTTTAGCATGTGTATTTTGTGTGCTCATTTACAACCGTCTC
GCGGCCGCGGGGATCCAGACATGATAAGATACATTGATGAGTTTGGACAAACCACAACTAGAATG
CAGTGAAAAAAATGCTTTATTTGTGAAATTTGTGATGCTATTGCTTTATTTGTAACCATTATAAGCT
GCAATAAACAAGTTAACAACAACAATTGCATTCATTTTATGTTTCAGGTTCAGGGGGAGGTGTGGG
AGGTTTTTTAGTCGACCTCGAGCAGTGTGGTTTTGCAAGAGGAAGCAAAAAGCCTCTCCACCCAGG
CCTGGAATGTTTCCACCCAAGTCGAAGGCAGTGTGGTTTTGCAAGAGGAAGCAAAAAGCCTCTCC
ACCCAGGCCTGGAATGTTTCCACCCAATGTCGAGCAACCCCGCCCAGCGTCTTGTCATTGGCGAAT
TCGAACACGCAGATGCAGTCGGGGCGGCGCGGTCCCAGGTCCACTTCGCATATTAAGGTGACGCG
TGTGGCCTCGAACACCGAGCGACCCTGCAGCCAATATGGGATCGGCCATTGAACAAGATGGATTG
CACGCAGGTTCTCCGGCCGCTTGGGTGGAGAGGCTATTCGGCTATGACTGGGCACAACAGACAAT
CGGCTGCTCTGATGCCGCCGTGTTCCGGCTGTCAGCGCAGGGGCGCCGGTTCTTTTTGTCAAGAC
CGACCTGTCCGGTGCCCTGAATGAACTGCAGGACGAGGCAGCGCGGCTATCGTGGCTGGCCACGA
CGGGCGTTCCTTGCGCAGCTGTGCTCGACGTTGTCACTGAAGCGGGAAGGGACTGGCTGCTATTGG
GCGAAGTGCCGGGGCAGGATCTCCTGTCATCTCACCTTGCTCCTGCCGAGAAAGTATCCATCATGG
CTGATGCAATGCGGCGGCTGCATACGCTTGATCCGGCTACCTGCCCATTCGACCACCAAGCGAAAC
ATCGCATCGAGCGAGCACGTACTCGGATGGAAGCCGGTCTTGTCGATCAGGATGATCTGGACGAA
GAGCATCAGGGGCTCGCGCCAGCCGAACTGTTCGCCAGGCTCAAGGCGCGCATGCCCGACGGCGA
GGATCTCGTCGTGACCCATGGCGATGCCTGCTTGCCGAATATCATGGTGGAAAATGGCCGCTTTTC
TGGATTCATCGACTGTGGCCGGCTGGGTGTGGCGGACCGCTATCAGGACATAGCGTTGGCTACCCG
TGATATTGCTGAAGAGCTTGGCGGCGAATGGGCTGACCGCTTCCTCGTGCTTTACGGTATCGCCGC
TCCCGATTCGCAGCGCATCGCCTTCTATCGCCTTCTTGACGAGTTCTTCTGAGGGGATCCGTCGACT
AGAGCTCGCTGATCAGCCTCGACTGTGCCTTCTAGTTGCCAGCCATCTGTTGTTTGCCCCTCCCCCG
TGCCTTCCTTGACCCTGGAAGGTGCCACTCCCACTGTCCTTTCCTAATAAAATGAGGAAATTGCAT
CGCATTGTCTGAGTAGGTGTCATTCTATTCTGGGGGGTGGGGTGGGGCAGGACAGCAAGGGGGAG
GATTGGGAAGACAATAGCAGGCATGCTGGGGAGAGATCTAGGAACCCCTAGTGATGGAGTTGGCCA
CTCCCTCTCTGCGCGCTCGCTCGCTCACTGAGGCCGCCCGGGCAAAGCCCGGGCGTCGGGCGACCTTT
GGTCGCCCGGCCTCAGTGAGCGAGCGAGCGCGCAGAGAGGGACTGGCCAACCCCCCCCCCCCCCCCC
C
SEQ ID NO: 25 gRNA7 spacer
ATTCCCCTGTATTATAAATGA
SEQ ID NO: 26: gRNA12 spacer
GAGTACCTCGGATTACTTAA

| | | | | |
|---|---|---|---|---|
| | Target/ | | | |
| Guide | Original | | | |
| # | gRNA | Cas9 | Spacer Sequence | (nt) |

First round screening

| | | | | |
|---|---|---|---|---|
| gAP1 | Dyst | SaCas9 | CTTTACTTTGTATTATGTAAA | |
| | (Intron51) | | (SEQ ID NO: 27) | 21 |
| gAP2 | Dyst | SaCas9 | TTTGAAATATTTTTGATATCT | |
| | (Intron51) | | SEQ ID NO: 28) | 21 |
| gAP3 | Dyst | SaCas9 | TTTAAGTAATCCGAGGTACTC | |
| | (Intron51) | | (SEQ ID NO: 29) | 21 |
| gAP4 | Dyst | SaCas9 | TTTAAATACATTGTCGTAATT | |
| | (Intron51) | | (SEQ ID NO: 30) | 21 |
| gAP5 | Dyst | SaCas9 | TACCTTAATTTTGACGTCACA | |
| | (Intron51) | | (SEQ ID NO: 31) | 21 |
| gAP6 | Dyst | SaCas9 | ATTTGACAGGTGAGAAATCTC | |
| | (Intron51) | | (SEQ ID NO: 32) | 21 |
| gAP7 | Dyst | SaCas9 | TCATTTATAATACAGGGGAAT | |
| | (Intron51) | | (SEQ ID NO: 33) | 21 |
| gAP8 | Dyst | SaCas9 | TTAAAGTCATTTATAATACAG | |
| | (Intron51) | | (SEQ ID NO: 34) | 21 |
| gAP9 | Dyst | SaCas9 | AAATAGACACTGAAGAAAGGG | |
| | (Intron51) | | (SEQ ID NO: 35) | 21 |
| gAP10 | Dyst | SaCas9 | CCCCAATTAAAATAAAATTTA | |
| | (Intron51) | | (SEQ ID NO: 36) | 21 |

Second round screening

| | | | | |
|---|---|---|---|---|
| gAP11 | g3 | SaCas9 | TAAGTAATCCGAGGTACTC | |
| | | | (SEQ ID NO: 37) | 19 |
| gAP12 | g3 | SaCas9 | TTAAGTAATCCGAGGTACTC | |
| | | | SEQ ID NO: 38) | 20 |
| gAP13 | g3 | SaCas9 | GTTTAAGTAATCCGAGGTACTC | |
| | | | (SEQ ID NO: 39) | 22 |
| gAP14 | g3 | SaCas9 | GGTTTAAGTAATCCGAGGTACTC | |
| | | | (SEQ ID NO: 40) | 23 |
| gAP15 | g6 | SaCas9 | TTGACAGGTGAGAAATCTC | |
| | | | (SEQ ID NO: 41) | 19 |
| gAP16 | g6 | SaCas9 | TTTGACAGGTGAGAAATCTC | |
| | | | (SEQ ID NO: 42) | 20 |
| gAP17 | g6 | SaCas9 | CATTTGACAGGTGAGAAATCTC | |
| | | | (SEQ ID NO: 43) | 22 |
| gAP18 | g6 | SaCas9 | TCATTTGACAGGTGAGAAATCTC | |
| | | | (SEQ ID NO: 44) | 23 |
| gAP19 | g7 | SaCas9 | ATTTATAATACAGGGGAAT | |
| | | | (SEQ ID NO: 45) | 19 |
| gAP20 | g7 | SaCas9 | CATTTATAATACAGGGGAAT | |
| | | | (SEQ ID NO: 5) | 20 |
| gAP21 | g7 | SaCas9 | GTCATTTATAATACAGGGGAAT | |
| | | | (SEQ ID NO: 6) | 22 |
| gAP22 | g7 | SaCas9 | AGTCATTTATAATACAGGGGAAT | |
| | | | (SEQ ID NO: 7) | 23 |
| gAP23 | scrambled | SaCas9 | GCACTACCAGAGCTAACTCA | |
| | | | (SEQ ID NO: 8) | 20 |

What is claimed is:

1. A CRISPR/Cas-based genome editing system comprising one or more vectors encoding a composition, the composition comprising:
(a) a guide RNA (gRNA) targeting a fragment of a mutant dystrophin gene;
(b) a Cas protein or a fusion protein comprises the Cas protein; and
(c) a donor sequence comprising a fragment of a wild-type dystrophin gene.

2. A CRISPR/Cas-based genome editing system comprising:
(a) a guide RNA (gRNA) targeting a fragment of a mutant dystrophin gene;
(b) a Cas protein or a fusion protein comprises the Cas protein; and
(c) a donor sequence comprising a fragment of a wild-type dystrophin gene.

3. The system of claim 1 or 2, wherein the fragment of the wild-type dystrophin gene is flanked by two gRNA spacers and/or PAM sequences.

4. The system of any one of claims 1-3, wherein the gRNA targets an intron that is juxtaposed with an exon of the mutant dystrophin gene, and wherein the exon is selected from exons 1-8, 10, 11, 12, 14, 16-22, 43-59, and 61-66 of the mutant dystrophin gene.

5. The system of any one of claims 1-3, wherein the donor sequence comprises an exon of the wild-type dystrophin gene or a functional equivalent thereof, and wherein the exon is selected from exons 1-8, 10, 11, 12, 14, 16-22, 43-59, and 61-66 of the wild-type dystrophin gene.

6. The system of claim 4, wherein the exon of the mutant dystrophin gene is mutated or at least partially deleted from the dystrophin gene, or wherein the exon of the mutant dystrophin gene is deleted and the intron is juxtaposed to where the deleted exon would be in a corresponding wild-type dystrophin gene.

7. The system of claim 4 or 5, wherein the exon is exon 52.

8. The system of any one of claims 1-7, wherein the gRNA binds and targets a polynucleotide sequence comprising:
a) SEQ ID NO: 17 or SEQ ID NO: 18;
b) a fragment of SEQ ID NO: 17 or SEQ ID NO: 18;
c) a complement of SEQ ID NO: 17 or SEQ ID NO: 18, or fragment thereof;
d) a nucleic acid that is substantially identical to SEQ ID NO: 17 or SEQ ID NO: 18, or complement thereof; or
e) a nucleic acid that hybridizes under stringent conditions to SEQ ID NO: 17 or SEQ ID NO: 18, complement thereof, or a sequence substantially identical thereto.

9. The system of any one of claims 1-8, wherein the gRNA comprises or is encoded by a polynucleotide sequence of SEQ ID NO: 19 or SEQ ID NO: 20, or a variant thereof.

10. The system of any one of claims 1-9, wherein the Cas protein is a Streptococcus pyogenes Cas9 protein or a Staphylococcus aureus Cas9 protein.

11. The system of any one of claims 1-10, wherein the Cas protein comprises an amino acid sequence of SEQ ID NO: 1, 2, 3, or 4.

12. The system of any one of claims 3-11, wherein the two gRNA spacers independently comprise a sequence selected from SEQ ID NO: 5-8 and 25-45.

13. The system of claim 12, wherein the two gRNA spacers are identical.

14. The system of claim 12, wherein the two gRNA spacers are different.

15. The system of any one of claims 3-14, wherein at least one of the two gRNA spacers comprises a sequence of SEQ ID NO: 25 or SEQ ID NO: 26.

16. The system of any one of claims 1-15, wherein the donor sequence comprises the polynucleotide of SEQ ID NO: 21 or SEQ ID NO: 22.

17. The system of any one of claims 1 and 3-16, wherein the vector is a viral vector.

18. The system of claim 17, wherein the vector is an Adeno-associated virus (AAV) vector.

19. The system of claim 18, wherein the AAV vector is AAV1, AAV2, AAV3, AAV4, AAV5, AAV6, AAV7, AAV8, AAV9, AAV-10, AAV-11, AAV-12, AAV-13, or AAVrh.74 vector.

20. The system of claim 18, wherein one of the one or more vectors comprises the polynucleotide sequence of SEQ ID NO: 23 or 24.

21. The system of any one of claims 1-20, wherein the molar ratio between gRNA and donor sequence is 1:1, or 1:15, or from 5:1 to 1:10, or from 1:1 to 1:5.

22. A recombinant polynucleotide encoding a donor sequence comprising a fragment of a wild-type dystrophin gene or a functional equivalent thereof, and wherein the fragment or functional equivalent thereof is flanked by two gRNA spacers.

23. The recombinant polynucleotide of claim 22, wherein the donor sequence comprises an exon of the dystrophin gene, and wherein the exon is selected from exons 1-8, 10, 11, 12, 14, 16-22, 43-59, and 61-66.

24. The recombinant polynucleotide of claim 22 or 23, wherein the recombinant polynucleotide comprises a sequence of SEQ ID NO: 23 or 24.

25. A vector comprising the recombinant polynucleotide of any one of claims 22-24.

26. The vector of claim 25, wherein the vector comprises a heterologous promoter driving expression of the recombinant polynucleotide.

27. A cell comprising the recombinant polynucleotide of any one of claims 22-24 or the vector of claim 25 or 26.

28. A composition for restoring dystrophin function in a cell having a mutant dystrophin gene, the composition comprising the system of any one of claims 1-21, the recombinant polynucleotide of any one of claims 22-24, or the vector of claim 25 or 26.

29. A kit comprising the system of any one of claims 1-21, the recombinant polynucleotide of any one of claims 22-24, or the vector of claim 25 or 26, or the composition of claim 28.

30. A method for restoring dystrophin function in a cell or a subject having a mutant dystrophin gene, the method comprising contacting the cell or the subject with the system of any one of claims 1-21, the recombinant polynucleotide of any one of claims 22-24, or the vector of claim 25 or 26, or the composition of claim 28.

31. The method of claim 30, wherein the dystrophin function is restored by insertion of exon 52 of the wild-type dystrophin gene.

32. The method of claim 30 or 31, wherein the subject is suffering from Duchenne Muscular Dystrophy.

33. A method for restoring dystrophin function in a cell or a subject having a disrupted dystrophin gene caused by one or more deleted or mutated exons, the method comprising contacting the cell or the subject with the system of any one of claims 1-21, the recombinant polynucleotide of any one of claims 22-24, or the vector of claim 25 or 26, or the composition of claim 28.

34. The method of claim 33, wherein dystrophin function is restored by inserting one or more wild-type exons of dystrophin gene corresponding to the one or more deleted or mutated exons.

35. The method of claim 34, wherein one of the deleted or mutated exons is exon 52.


SEQUENCE LISTING (45 sequences)

SEQ ID NO: 1 - protein, 1368 aa, Streptococcus pyogenes [sequence omitted]

SEQ ID NO: 2 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 3 - protein, 1368 aa, Artificial Sequence
  Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 4 - protein, 1368 aa, Artificial Sequence
  Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 5 - DNA, 20 bp, Artificial Sequence
  Synthetic
catttataatacaggggaat

SEQ ID NO: 6 - DNA, 22 bp, Artificial Sequence
  Synthetic
gtcatttataatacaggggaat

SEQ ID NO: 7 - DNA, 23 bp, Artificial Sequence
  Synthetic
agtcatttataatacaggggaat

SEQ ID NO: 8 - DNA, 20 bp, Artificial Sequence
  Synthetic
gcactaccagagctaactca

SEQ ID NO: 9 - DNA, 6 bp, Artificial Sequence
  Synthetic
attcct

SEQ ID NO: 10 - DNA, 3 bp, Artificial Sequence
  Synthetic
  misc_feature (1)..(1): n is a, c, g, or t
ngg

SEQ ID NO: 11 - DNA, 6 bp, Artificial Sequence
  Synthetic
  misc_feature (1)..(3): n is a, c, g, or t
  r (4)..(4): r is a, or g
  r (5)..(5): r is a, or g
nnnrrt

SEQ ID NO: 12 - DNA, 5 bp, Artificial Sequence
  Synthetic
  misc_feature (1)..(2): n is a, c, g, or t
  r (4)..(4): r is a, or g
  r (5)..(5): r is a, or g
nngrr

SEQ ID NO: 13 - DNA, 6 bp, Artificial Sequence
  Synthetic
  misc_feature (1)..(2): n is a, c, g, or t
  r (4)..(4): r is a, or g
  r (5)..(5): r is a, or g
  misc_feature (6)..(6): n is a, c, g, or t
nngrrn

SEQ ID NO: 14 - DNA, 6 bp, Artificial Sequence
  Synthetic
  misc_feature (1)..(2): n is a, c, g, or t
  r (4)..(4): r is a, or g
  r (5)..(5): r is a, or g
nngrrt

SEQ ID NO: 15 - DNA, 6 bp, Artificial Sequence
  Synthetic
  misc_feature (1)..(2): n is a, c, g, or t
  r (4)..(4): r is a, or g
  r (5)..(5): r is a, or g
  v (6)..(6): v is a, c, or g
nngrrv

SEQ ID NO: 16 - DNA, 3 bp, Artificial Sequence
  Synthetic
  misc_feature (1)..(1): n is a, c, g, or t
nga

SEQ ID NO: 17 - DNA, 21 bp, Artificial Sequence
  Synthetic
tcatttataatacaggggaat

SEQ ID NO: 18 - DNA, 20 bp, Artificial Sequence
  Synthetic
ttaagtaatccgaggtactc

SEQ ID NO: 19 - DNA, 97 bp, Artificial Sequence
  Synthetic
tcatttataatacaggggaatgttttagtactctggaaacagaatctactaaaacaaggcaaaatgccgtgtttatctcgtcaacttgttggcgaga

SEQ ID NO: 20 - DNA, 96 bp, Artificial Sequence
  Synthetic
ttaagtaatccgaggtactcgttttagtactctggaaacagaatctactaaaacaaggcaaaatgccgtgtttatctcgtcaacttgttggcgaga

SEQ ID NO: 21 - DNA, 118 bp, Artificial Sequence
  Synthetic
gcaacaatgcaggatttggaacagaggcgtccccagttggaagaactcattaccgctgcccaaaatttgaaaaacaagaccagcaatcaagaggctagaacaatcattacggatcgaa

SEQ ID NO: 22 - DNA, 470 bp, Artificial Sequence
  Synthetic
gttaaattgttttctataaacccttatacagtaacatcttttttatttctaaaagtgttttggctggtctcacaattgtactttactttgtattatgtaaaaggaatacacaacgctgaagaaccctgatactaagggatatttgttcttacaggcaacaatgcaggatttggaacagaggcgtccccagttggaagaactcattaccgctgcccaaaatttgaaaaacaagaccagcaatcaagaggctagaacaatcattacggatcgaagtaagttttttaacaagcatgggacacacaaagcaagatgcatgacaagtttcaataaaaacttaagttcatatatccccctcacatttataaaaataatgtgaaataattgtaaatgataacaattgtgctgagattttcagtccataatgttaccttttaataaatgaatgtaattccattgaatagaagaaatac

SEQ ID NO: 23 - DNA, 4294 bp, Artificial Sequence
  Synthetic
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtaccttcctagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtcatttataatacaggggaatgttttagtactctggaaacagaatctactaaaacaaggcaaaatgccgtgtttatctcgtcaacttgttggcgagatttttttctagacccagctttcttgtacaaagttggcgtttaaacattcctattcccctgtattataaatgagttaaattgttttctataaacccttatacagtaacatcttttttatttctaaaagtgttttggctggtctcacaattgtactttactttgtattatgtaaaaggaatacacaacgctgaagaaccctgatactaagggatatttgttcttacaggcaacaatgcaggatttggaacagaggcgtccccagttggaagaactcattaccgctgcccaaaatttgaaaaacaagaccagcaatcaagaggctagaacaatcattacggatcgaagtaagttttttaacaagcatgggacacacaaagcaagatgcatgacaagtttcaataaaaacttaagttcatatatccccctcacatttataaaaataatgtgaaataattgtaaatgataacaattgtgctgagattttcagtccataatgttaccttttaataaatgaatgtaattccattgaatagaagaaatacattcctattcccctgtattataaatgagctagctcggagagacgacatctagaattaaactgtcactatcgattactaattttttgctcataatagaagcagcgattaaaggaatagaatcaacagttccagtaacatctcttagtgcatacatttttttatcagcaggaacaatatcatctgactgacctgtgatgctcattccaacttcattaattgttttaatgaatttttctttagataattcacttgttcaacctttacatgactctaatttatcaatagtaccaccagttactccaagtcctctaccagaaagtttacaaacctttactccataacttgcaactaacggactatatactaaacttgttttgtctccaactccgccagttgaatgcttatcagcttttaaacctgtaacctcactgacatcataaacatatcctgattcaacataagattgggttaatgctaaagtttctgctttggtcattccattaaaataaaccgccatagcaaaagcagccatttgataatctgttacattatttttaacgtaactgtttatcaatcatttaatttcttcagctgataattctattgaatgtttctttttttctataatttcactaaaactgtagttcataagtctccttttgtaagagtgcacaatatttacaccattactctttctactatattataatagaatagacatataaaaaacataaggagtacaaatggtttttgataaaaataacaaagtttatagtgaatgaataaatagccaaaaattggatgatgagttgaaaagccttttagtaaatgctactgatgatgaattgcatgcagcatttgaaggaatagagttagaatttggaacagcaggtataagaggtattcttggagcaggacctggaagatttaacgtttacactgttaaaaaagttactattgcatttgcagaattattaaaacaaaattacccaaataggttgaatgatggaatagttgttggtcatgataaccgtcataattctaaacagtttgcaaaagttgtagccgaagttttatcaagcttgtgaaatagctgttgaagctggattagaatttgttaaaacatcaacaggattttcaaaatcaggtgcaacatttgaagatgttaaactaatgaagtcagttgttaaagacaatgctttagttaaagcagctggtggagttagaacatttgaagatgctcaaaaaatgattgaagcaggagctgaccgcttaggaacaagtggtggagtagctattattaaaggtgaagaaaacaacgcgagttactaaaactagcgtttttttattttgctcatttttattaaaagtttgcaaaaaggaacataaaaattctaattattgatactaaagttattaaaaagaagattttggttgattttataaaggtcatagaatataatattttagcatgtgtattttgtgtgctcatttacaaccgtctcgcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgacctcgagcagtgtggttttgcaagaggaagcaaaaagcctctccacccaggcctggaatgtttccacccaagtcgaaggcagtgtggttttgcaagaggaagcaaaaagcctctccacccaggcctggaatgtttccacccaatgtcgagcaaccccgcccagcgtcttgtcattggcgaattcgaacacgcagatgcagtcggggcggcgcggtcccaggtccacttcgcatattaaggtgacgcgtgtggcctcgaacaccgagcgaccctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccc

SEQ ID NO: 24 - DNA, 4291 bp, Artificial Sequence
  Synthetic
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtaccttcctagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgttaagtaatccgaggtactcgttttagtactctggaaacagaatctactaaaacaaggcaaaatgccgtgtttatctcgtcaacttgttggcgagatttttttctagacccagctttcttgtacaaagttggcgtttaaacattcctgagtacctcggattacttaagttaaattgttttctataaacccttatacagtaacatcttttttatttctaaaagtgttttggctggtctcacaattgtactttactttgtattatgtaaaaggaatacacaacgctgaagaaccctgatactaagggatatttgttcttacaggcaacaatgcaggatttggaacagaggcgtccccagttggaagaactcattaccgctgcccaaaatttgaaaaacaagaccagcaatcaagaggctagaacaatcattacggatcgaagtaagttttttaacaagcatgggacacacaaagcaagatgcatgacaagtttcaataaaaacttaagttcatatatccccctcacatttataaaaataatgtgaaataattgtaaatgataacaattgtgctgagattttcagtccataatgttaccttttaataaatgaatgtaattccattgaatagaagaaatacattcctgagtacctcggattacttaagctagctcggagagacgacatctagaattaaactgtcactatcgattactaattttttgctcataatagaagcagcgattaaaggaatagaatcaacagttccagtaacatctcttagtgcatacatttttttatcagcaggaacaatatcatctgactgacctgtgatgctcattccaacttcattaattgttttaatgaatttttctttagataattcacttgttcaacctttacatgactctaatttatcaatagtaccaccagttactccaagtcctctaccagaaagtttacaaacctttactccataacttgcaactaacggactatatactaaacttgttttgtctccaactccgccagttgaatgcttatcagcttttaaacctgtaacctcactgacatcataaacatatcctgattcaacataagattgggttaatgctaaagtttctgctttggtcattccattaaaataaaccgccatagcaaaagcagccatttgataatctgttacattatttttaacgtaactgtttatcaatcatttaatttcttcagctgataattctattgaatgtttctttttttctataatttcactaaaactgtagttcataagtctccttttgtaagagtgcacaatatttacaccattactctttctactatattataatagaatagacatataaaaaacataaggagtacaaatggtttttgataaaaataacaaagtttatagtgaatgaataaatagccaaaaattggatgatgagttgaaaagccttttagtaaatgctactgatgatgaattgcatgcagcatttgaaggaatagagttagaatttggaacagcaggtataagaggtattcttggagcaggacctggaagatttaacgtttacactgttaaaaaagttactattgcatttgcagaattattaaaacaaaattacccaaataggttgaatgatggaatagttgttggtcatgataaccgtcataattctaaacagtttgcaaaagttgtagccgaagttttatcaagcttgtgaaatagctgttgaagctggattagaatttgttaaaacatcaacaggattttcaaaatcaggtgcaacatttgaagatgttaaactaatgaagtcagttgttaaagacaatgctttagttaaagcagctggtggagttagaacatttgaagatgctcaaaaaatgattgaagcaggagctgaccgcttaggaacaagtggtggagtagctattattaaaggtgaagaaaacaacgcgagttactaaaactagcgtttttttattttgctcatttttattaaaagtttgcaaaaaggaacataaaaattctaattattgatactaaagttattaaaaagaagattttggttgattttataaaggtcatagaatataatattttagcatgtgtattttgtgtgctcatttacaaccgtctcgcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgacctcgagcagtgtggttttgcaagaggaagcaaaaagcctctccacccaggcctggaatgtttccacccaagtcgaaggcagtgtggttttgcaagaggaagcaaaaagcctctccacccaggcctggaatgtttccacccaatgtcgagcaaccccgcccagcgtcttgtcattggcgaattcgaacacgcagatgcagtcggggcggcgcggtcccaggtccacttcgcatattaaggtgacgcgtgtggcctcgaacaccgagcgaccctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccc

SEQ ID NO: 25 - DNA, 21 bp, Artificial Sequence
  Synthetic
attcccctgtattataaatga

SEQ ID NO: 26 - DNA, 20 bp, Artificial Sequence
  Synthetic
gagtacctcggattacttaa

SEQ ID NO: 27 - DNA, 21 bp, Artificial Sequence
  Synthetic
ctttactttgtattatgtaaa

SEQ ID NO: 28 - DNA, 21 bp, Artificial Sequence
  Synthetic
tttgaaatatttttgatatct

SEQ ID NO: 29 - DNA, 21 bp, Artificial Sequence
  Synthetic
tttaagtaatccgaggtactc

SEQ ID NO: 30 - DNA, 21 bp, Artificial Sequence
  Synthetic
tttaaatacattgtcgtaatt

SEQ ID NO: 31 - DNA, 21 bp, Artificial Sequence
  Synthetic
taccttaattttgacgtcaca

SEQ ID NO: 32 - DNA, 21 bp, Artificial Sequence
  Synthetic
atttgacaggtgagaaatctc

SEQ ID NO: 33 - DNA, 21 bp, Artificial Sequence
  Synthetic
tcatttataatacaggggaat

SEQ ID NO: 34 - DNA, 21 bp, Artificial Sequence
  Synthetic
ttaaagtcatttataatacag

SEQ ID NO: 35 - DNA, 21 bp, Artificial Sequence
  Synthetic
aaatagacactgaagaaaggg

SEQ ID NO: 36 - DNA, 21 bp, Artificial Sequence
  Synthetic
ccccaattaaaataaaattta

SEQ ID NO: 37 - DNA, 19 bp, Artificial Sequence
  Synthetic
taagtaatccgaggtactc

SEQ ID NO: 38 - DNA, 20 bp, Artificial Sequence
  synthetic
ttaagtaatccgaggtactc

SEQ ID NO: 39 - DNA, 22 bp, Artificial Sequence
  Synthetic
gtttaagtaatccgaggtactc

SEQ ID NO: 40 - DNA, 23 bp, Artificial Sequence
  Synthetic
ggtttaagtaatccgaggtactc

SEQ ID NO: 41 - DNA, 19 bp, Artificial Sequence
  Synthetic
ttgacaggtgagaaatctc

SEQ ID NO: 42 - DNA, 20 bp, Artificial Sequence
  Synthetic
tttgacaggtgagaaatctc

SEQ ID NO: 43 - DNA, 22 bp, Artificial Sequence
  Synthetic
catttgacaggtgagaaatctc

SEQ ID NO: 44 - DNA, 23 bp, Artificial Sequence
  Synthetic
tcatttgacaggtgagaaatctc

SEQ ID NO: 45 - DNA, 19 bp, Artificial Sequence
  Synthetic
atttataatacaggggaat